(19) United States
(12) Patent Application Publication
(10) Pub. No.: US 2025/0034519 A1
(43) Pub. Date: Jan. 30, 2025

(54) HYDROGELATED CELLS

(71) Applicants: The Regents of the University of California, Oakland, CA (US); Academia Sinica, Taipei City (TW)

(72) Inventors: Cheemeng TAN, Davis, CA (US); Luis E. CONTRERAS LLANO, Davis, CA (US); Che-Ming J. HU, Taipei (TW)

(21) Appl. No.: 18/896,384

(22) Filed: Sep. 25, 2024

Related U.S. Application Data
(63) Continuation of application No. PCT/US2023/016705, filed on Mar. 29, 2023.
(60) Provisional application No. 63/325,367, filed on Mar. 30, 2022.

(51) Int. Cl. C12N 5/07; C12N 13/00; G01N 33/483

(52) U.S. Cl. CPC C12N 5/06; C12N 13/00; G01N 33/4833; C12N 2501/999

(57) ABSTRACT

Assembly of a synthetic polymer network inside cells is described that renders the cells incapable of dividing. The resulting cells can retain functions, including for example, cellular metabolism, motility, protein synthesis, and compatibility with genetic circuits. The cells can also acquire new abilities to resist stressors that otherwise kill natural cells.

### CROSS-REFERENCE TO RELATED PATENT APPLICATIONS

The present application is a continuation of International Application No. PCT/US2023/016705, filed Mar. 29, 2023, which claims benefit of priority to U.S. Provisional Patent Application No. 63/325,367, filed Mar. 30, 2022, each of which is incorporated by reference for all purposes.

### STATEMENT AS TO RIGHTS TO INVENTIONS MADE UNDER FEDERALLY SPONSORED RESEARCH AND DEVELOPMENT

This invention was made with government support under GM142788 and EB025938 awarded by the National Institutes of Health. The government has certain rights in the invention.

### BACKGROUND OF THE INVENTION

### Introduction

Synthetic biology has made major strides towards the holy grail of fully programable bio-micromachines capable of sensing and responding to defined stimuli regardless of their environmental context. A common type of bio-micromachines is created by genetically modifying living cells (Nielsen and Keasling, Cell. 164, 1185-1197 (2016)). Living cells possess the unique advantage of being highly adaptable and versatile (Xie and Fussenegger, Nat. Rev. Mol. Cell Biol. 19, 507-525 (2018). To date, living cells have been successfully repurposed for a wide variety of applications, including living therapeutics (Charbonneau et al., Nat. Commun. 11, 1-11 (2020)), bioremediation (Dvořák et al., Biotechnol. Adv. 35, 845-866 (2017)), and drug and gene delivery (Din et al., Nature. 536, 81-85 (2016); N. S. Forbes, Nat. Rev. Cancer. 10, 785-794 (2010)). However, the resulting synthetic living cells are challenging to control due to their continuous adaption and evolving cellular context. Application of these autonomously replicating organisms often requires tailored biocontainment strategies (A. J. Simon, A. D. Ellington, F1000Research. 5, 1-8 (2016); Rovner, Nature. 518, 89-93 (2015); Mandell et al., Nature. 518, 55-60 (2015)), which can raise logistical hurdles and safety concerns.

In contrast, non-living synthetic cells, notably artificial cells (Zhang et al., Trends Biotechnol. 26, 14-20 (2008); Ding et al., Life. 4, 1092-1116 (2014)), can be created using synthetic materials, such as polymers or phospholipids. Meticulous engineering of materials enables defined partitioning of bioactive agents, and the resulting biomimetic systems possess advantages including predictable functions, tolerance to certain environmental stressors, and ease of engineering (Ding et al., ACS Appl. Mater. Interfaces. 10, 30137-30146 (2018); Tan et al., Nat. Nanotechnol. 8, 602-608 (2013)). Non-living cell-mimetic systems have been employed to deliver anticancer drugs (Briolay et al., Mol. Cancer. 20, 1-24 (2021)), promote antitumor immune responses (Park et al., Nat. Mater. 11, 895-905 (2012)), communicate with other cells (Robinson et al., Curr. Opin. Chem. Biol. 64, 165-173 (2021)), mimic immune cells (Hasani-Sadrabadi et al., Adv. Mater. 30, 1870159 (2018); Lin et al., Adv. Mater. 33, 1-12 (2021)), and perform photosynthesis (Berhanu et al., Nat. Commun. 10 (2019), doi: 10.1038/s41467-019-09147-4). Compared to living cells, however, current non-living systems have limited biochemical complexities and biological functions owing to the constraints of bottom-up engineering. Continuing efforts are devoted to advancing synthetic cells for enhanced environmental responsiveness and cell-like capabilities.

### BRIEF SUMMARY OF THE INVENTION

The disclosure provides, e.g., a metabolically-active cell comprising a cross-linked hydrogel within the cell in sufficient amount to prevent cell replication. In some embodiments, the hydrogel comprises monosaccharide or polysaccharide monomer subunits and wherein the hydrogel is a homopolymer or co-polymer. In some embodiments, the hydrogel comprises substituted or unsubstituted poly (ethylene glycol) monomer subunits. In some embodiments, the hydrogel comprises poly (dimethyl siloxane) (PDMS), poly (ethylene oxide) (PEO), poly (vinyl alcohol) (PVA), poly (propylene fumarate) (PPF), alginate, guanosine mono phosphate (GMP), cyclodextrin (CD), fibrin, collagen, polypeptides, decellularized extracellular matrix, or nucleic acids.

In some embodiments, the hydrogel is substituted. In some embodiments, the hydrogel is conjugated to a metal, bioactive or therapeutic molecule, drug, nanoparticle, nucleic acid, or polypeptide. In some embodiments, the bioactive molecule is an anti-cancer molecule.

In some embodiments, the hydrogel has a density of 1-10% (w/w) in the cell.

In some embodiments, the cells are prokaryotic cells. In some embodiments, the prokaryotic cells are gram negative bacteria. In some embodiments, the gram-negative bacteria are selected from the genera consisting of Escherichia, Proteus, Enterobacter, Klebsiella, Citrobacter, Yersinia, Shigella, and Salmonella. In some embodiments, the cells are eukaryotic cells. In some embodiments, the cells are eukaryotic cells are yeast or plant or mammalian (e.g., human) cells. In some embodiments, the eukaryotic cells are Saccharomyces cerevisiae cells. In some embodiments, the mammalian cells are HeLa, HEK293, or SH-SY5Y cells.

In some embodiments, the cells further comprise at least one heterologous nucleic acid. In some embodiments, the heterologous nucleic acid encodes a protein. In some embodiments, the protein is an enzyme.

In some embodiments, the cell has been modified to have a reduced amount of one or more nuclease, protease and protein involved in stress response compared to a native cell. In some embodiments, the cell is contacted with a heterologous cryoprotectant.

In some embodiments, the cell is modified with a heterologous molecule that directs flux of ATP and/or NADH.

Also provided is a method comprising administering the cells as described above or elsewhere herein to an animal. In some embodiments, the animal is human.

Also provided is a method of assaying a cellular activity of the cells as described above or elsewhere herein. In some embodiments, the method comprises measuring at least one activity of the cells. In some embodiments, the measuring comprises contacting the cells with an agent and measuring the effect of the agent on the activity of the cells. In some embodiments, the activity is selected from the group consisting of cellular motility, intracellular redox (reduction/oxidation) state, membrane fluidity, and protein expression capabilities.

Also provided is a method of generating the cell as described above or elsewhere herein. In some embodiments, the method comprises providing dividing cells; introducing monomer units of a hydrogel into the cells; and causing the polymerization inducer to initiate formation in the cells of a hydrogel formed from the monomer units thereby forming a mixture of cells comprising the hydrogel. In some embodiments, the method further comprises introducing a polymerization inducer into the cells before, after or simultaneously with the introducing of the monomer units. In some embodiments, the polymerization inducer is activated by light of a specific wavelength and the causing comprising exposing the cells to light of the specific wavelength. In some embodiments, the monomer subunits comprise one or more acrylate moieties and the polymerization inducer is selected from the group consisting of 2-hydroxyl-4′-(2-hydroxyethoxy)-2-methylpropiophenone, Irgacure 2959, Eosin-Y, and lithium phenyl-2,4,6-tri-methylbenzoylphosphinate.

In some embodiments, the monomer subunits comprise substituted or unsubstituted poly (ethylene glycol) monomer subunits. In some embodiments, the substituted or unsubstituted poly (ethylene glycol) monomer subunits comprise poly (ethylene glycol) diacrylate, poly (ethylene glycol) thiol poly (ethylene glycol) vinyl sulfone, alginate, guanosine mono phosphate (GMP), cyclodextrin (CD), fibrin, collagen, polypeptides, decellularized extracellular matrix, or nucleic acids.

In some embodiments, the monomer subunits comprise poly (dimethyl siloxane) (PDMS), poly (ethylene oxide) (PEO), poly (vinyl alcohol) (PVA), or poly (propylene fumarate) (PPF).

In some embodiments, the introducing comprises exposing the cells to a freeze/thaw cycle in the presence of the monomer units and the polymerization inducer.

In some embodiments, the method further comprises contacting the mixture of cells with a replication-specific toxin and/or antibiotics, thereby killing cells in the mixture capable of replicating.

In some embodiments, the method further comprises contacting the cell with a heterologous cryoprotectant during the providing, introducing and/or causing.

### BRIEF DESCRIPTION OF THE DRAWINGS

FIG. 1A-I: Engineering Cyborg Cells through intracellular hydrogelation.

FIG. 1A) Graphic representation of a Cyborg Cell highlighting its characteristics. Cyborg Cells do not divide, preserve metabolic and protein-synthesis activities, maintain membrane fluidity, and gain new resistance to environmental stressors.

FIG. 1B) Schematic illustrating the procedure to hydrogelate E. coli cells. 1) Mix the hydrogel buffer with an exponentially growing culture of the desired E. coli strain. 2) Make the hydrogel buffer permeate the bacterial membrane through a freeze and thaw cycle (−80 to 37° C.). 3) Eliminate replicating cells using a high concentration of Carbenicillin.

FIG. 1C) Membrane solubilization using 1% SDS to evaluate successful bacterial hydrogelation of E. coli BL21 (DE3). Top panels: Representative microscope images of the bacteria infused with hydrogel components and treated (+) and untreated (−) with 1% SDS. Bottom panels: Histogram of single-cell fluorescence intensity. Non-hydrogelated bacteria show a decrease in fluorescence intensity caused by the escape of Fluorescein DA after SDS treatment. In contrast, Cyborg Cells maintain green fluorescence intensity after 1% SDS treatment. (Scale bar=5 μm, n=3 independent experiments). See Methods Section M3, and FIG. 6 for replicates.

FIG. 1D) CFU counting assays confirm that Cyborg Cells cannot replicate. Top panel: CFU counts of hydrogelated and non-hydrogelated bacteria under different conditions on Day 1. Bottom panel: CFU counts of Cyborg Cells and Wild Type bacteria (WT, E. coli BL21 (DE3) across 7 days. (Error bar=SD, n=3 independent experiments).

FIG. 1E) Cyborg Bacteria Cells preserve metabolic activity. Cyborg Cells created using the strain E. coli BL21 (DE3) show comparable levels of metabolic activity according to an assay measuring reduction capacity inside the cell. See Methods M4. Data are presented as mean values. (Error bar=SD, n=3 independent experiments). Standard two-tail t-test.

FIG. 1F) Fluorescence recovery after photobleaching (FRAP) assay shows the preservation of membrane fluidity in Cyborg Cells. The membrane of Cyborg, Wild Type, and fixed cells. (Scale bar=2 μm, n=3 independent experiments). See Methods Section M7 and FIG. 10.

FIG. 1G&H) Cyborg E. coli MG1655 (G) and E. coli Nissle 1917 (H) cells. Top panels: Fluorescence microscopy images of hydrogelated bacteria. The hydrogel is labeled with Fluorescein (Methods Section M2, FIG. 11) (Scale bar=5 μm, n=3 independent experiments). Bottom panels: Metabolic activity of Cyborg & Wild Type cells (FIG. 1) (n=3 independent experiments).

FIG. 1I) Cyborg E. coli BL21 (DE3) and MG1655 preserve motility similar to untreated cells. Sequential timelapse images of Cyborg and untreated cells showing similar motility patterns. We followed individual cells across 100 Frames (˜5 s) See Supplementary Videos 1-4 (Scale bar=5 μm, n=3 independent experiments).

FIG. 2A-E: Protein expression and proteome characterization of Cyborg Cells

FIG. 2A) Cyborg Bacterial Cells express mOrange in response to IPTG induction. Fluorescence microscopy images of Cyborg Cells derived from the strain E. coli BL21 (DE3) pIURKL-mOrange pLysS after 12 h incubation with and without 1 mM IPTG. (Scale bar=5 μm, n=3 independent experiments).

FIG. 2B) Single-cell tracking of mOrange expressing Cyborg Cells. Cyborg cells did not grow but expressed mOrange after 8 h of IPTG induction. (Scale bar=5 μm, Methods Section M5).

FIG. 2C) Cyborg Cells and Wild Type Bacteria show comparable mOrange expression. Expression levels of mOrange after 12 h incubation with (+) and without (−) IPTG (Methods Section M11). See FIG. 13 for the continuous tracking of the reaction and for optical density measurements of the samples during this experiment. (Error bar=SD, n=4 independent experiments). Standard two-tail t-test.

FIG. 2D) Principal component analysis (PCA) shows the grouping of our different samples based on their protein profile.

FIG. 2E) Total log difference between the protein intensities of each sample calculated as the average of each functional group and compared against the abundance of the proteins in our Wild Type control. The colorbar indicates the color code for the value of total log difference.

FIG. 3A-O: Cyborg Cells can be functionalized using synthetic biology parts.

FIG. 3A) Schematic of the Marionette-Pro strain and its sensor array.

FIG. 3B-M) Response of Wild Type Controls (Left Panel) and Cyborg Cells (Right Panel) to the small molecule activating YFP expression in each strain. Wild Type and Cyborg Cells uninduced (−) and induced (+) using: B) 25 μM DAPG (2,4-diacetylphloroglucinol). C) 100 μM Cuma (Cuminic acid). D) 10 uM OC6 (30C6-AHL). E) 100 μM Van (Vanillic acid). F) 1 mM IPTG (Isopropyl-β-d-thiogalactoside). G) 200 nM aTc (anhydrotetracycline HCL). H) 4 mM Ara (L-arabinose). I) 10 mM Cho (choline chloride). J) 1 mM Nar (naringenin). K) 1 mM DHBA (3,4-Dihydroxy benzoic acid). L) 100 uM Sal (sodium salicylate). M) 10 μM OHC14 (30HCl4: 1-AHL). Each schematic on the left of each plot shows the activation (black arrow) of each promoter by the corresponding small molecule inducer (colored circles). (n=4 independent experiments).

FIG. 3N) Expression rate of each Wild Type and Cyborg Cell strain functionalized with different synthetic circuits. Filled green bar=Cyborg Cells without inducer. Open green bars=Cyborg Cells with inducer. Filled black bar=Wild Type cells without inducer. Open black bars=Wild Type cells with inducer. CC=Cyborg Cells. WT=Wild Type Cells. (Error bars=SD, n=4 independent experiments). All uninduced and induced pairs show significant differences, except as indicated (n.s.). Only the significantly different induced expression rates are highlighted (*).

FIG. 3O) Optical density changes (OD600 nm) over 10 hours of each circuit. (Error bars SD, n=4 independent experiments). See Methods Section M10.

FIG. 4A-C: Cyborg cells gain new non-native functions. FIG. 4A) Cyborg E. coli (Migula) Castellani and Chalmers remain stable after hydrogen peroxide (H₂O₂) treatment (10% w/w; 3M). Wild Type cells are lysed under the treatment with H₂O₂. All cells were fixed with 4% paraformaldehyde and then stained with DAPI (10 mg/mL) (Blue color). Representative images (n=3 independent experiments). FIG. 4B) Cyborg E. coli BL21 (DE3) Cells resist D-Cycloserine treatment. Cyborg Cells remain stable and express fluorescent proteins. Wild Type cells (WT) were lysed. Cyborg Cells remain stable and are capable of mOrange expression when incubated in media containing 200 (μg/mL) D-Cycloserine. Cyborg cells remain stable and express fluorescent proteins. Wild Type cells (WT) when incubated under the same conditions were lysed. (n=3 independent experiments). See FIG. 17. FIG. 4C) Cyborg E. coli BL21 (DE3) Cells remain stable in media with high pH. Cyborg Cells express mOrange when incubated in media at pH 7-9. Under our experimental conditions, at pH 8, Wild Type cells form filaments and stop expressing the fluorescent protein reporter. At pH 9, the cells are lysed. (n=3 independent experiments). See FIG. 18.

FIG. 5A-G: Cyborg Cells are capable of cancer cell invasion.

FIG. 5A) Schematic of Cyborg Cells expressing mOrange and Invasin (inv+) invading cancer cells. This uptake is facilitated by the binding of invasin and B1-integrins displayed on the membrane of cancer cells.

FIG. 5B) Confocal microscopy images of SY-SY5Y cells co-incubated with Cyborg E. coli BL21 (DE3) Cells expressing (+inv) and not expressing (−inv) invasin. Controls are SY-SY5Y cells stained with Hoechst with no Cyborg Cells. All Cyborg Cells express mOrange (orange) and all SY-SY5Y cells are stained with Hoechst (blue) (Scalebar=10 mm, n=2 independent experiments). See Methods Section M14.

FIG. 5C) Representative image of Cyborg Cells expressing mOrange incubated with SH-SY5Y cells. The images were obtained through confocal microscopy (Methods Section M14). They were fake colorized for clarity and to facilitate blind counting of cyborg cells invading mammalian cells. (Scale bar=10 mm, n=2 independent experiments).

FIG. 5D) Representative image of Cyborg Cells expressing mOrange and Invasin incubated with SH-SY5Y cells. The images were obtained through confocal microscopy (Methods Section M14) and fake colorized for clarity and to help the analysis through blind counting of cy borg cells invading mammalian cells. (Scale bar=10 mm, n=3 independent experiments).

FIG. 5E) Cross-sectional Z-stack images obtained by confocal microscopy showing Cyborg Cells expressing mOrange and Invasin incubated with SH-SY5Y cells. Blue: cell nuclei of cells stained with Hoechst dye. Orange: mOrange of Cyborg Cells. Grey: Bright field. See Methods Section M14. (Scale bar=10 mm, n=2 independent experiments).

FIG. 5F) Ratio of SH-SY5Y cells invaded by Cyborg Cells (inv=Invasin). Error bar=SEM (n=12). See Methods Section M14, and FIG. 19.

FIG. 5G) Representative images of Wild Type, Cyborg, and Fixed Cells, incubated with HeLa cells. Methods Section M15, (Scale bars=10 & 5 mm).

FIG. 6A-B: Characterization of the PEG-700 dyacrilate hydrogel matrix in vivo and in vitro. A) Replicate images of the SDS detergent treatment of Cyborg and non-hydrogelated E. coli cells. (Scalebar=5 mm. See Methods Section M3). B) CryoSEM image of the loose porous structure of hydrogel scaffold generated by 5% PEG diacrylate. (Scalebar=5 mm. See Methods Section M6).

FIG. 7A-D: Optimization of the intracellular hydrogelation protocol. Optimization of: FIG. 7A). PEG-DA concentration FIG. 7B) UV irradiation energy. FIG. 7C) Reaction volume FIG. 7D). Cell mass to volume ratio. Representative images of 3 independent experiments. Scalebar=5 mm.

FIG. 8A-B: Cyborg Cells are generated under optimal hydrogelation conditions. 8A) Optimal PEG-DA % and UV crosslinking time. Orange region highlights the parameters that generate Cyborg E. coli BL21 (DE3) Cells. Hydrogelation conditions were screened in 96-well plates. 8B) Metrics used for identifying Cyborg Cells. Cyborg cells should not replicate (first column) but should preserve metabolic activity (2^{nd} column, NADP reduction to NADPH) and protein-synthesis (3^{rd} column) activities. mOrange expressing E. coli BL21 (DE3) cells under the different conditions listed were characterized to obtain a characteristic phenotype for Cyborg Cells using four different parameters: Cell division, metabolic activity, and protein expression, and characteristic morphology. Scalebar=5 mm.

FIG. 9: Metabolic activity of Cyborg E. coli BL21 (DE3) Cells. Metabolic activity of Wild Type and Cyborg E. coli BL21 (DE3) Cells. See Methods Section M4. Error bars=SD (n=4 independent experiments).

FIG. 10A-D: Replicates of the FRAP assays on Living, Cyborg and Fixed Cells. 10A) Images of the FRAP experiments performed in living E. coli cells showing the Pre, Post and Bleached stages of the experiment. 10B) Images of the FRAP experiments performed in Cyborg E. coli cells showing the Pre, Post and Bleached stages of the experiment. 10C) Images of the FRAP experiments performed in fixed (Non-hydrogelated) E. coli cells showing the Pre, Post and Bleached stages of the experiment. Scalebar=2 mm. 10D) FRAP curves showing the percentage of fluorescent intensity (a.u) compared to the pre-Bleached samples of Live (green line), Cyborg (red line), and Fixed (grey line) cells. Images were taken at timepoints Os (Pre-Bleach), 8 s (Bleached), and 120 s (Post-Bleached). See Methods Section M7. Error bars=SEM (n=3 independent experiments).

FIG. 11A-B: Replicates showing the creation of Cyborg E. coli MG1655 & Nissle 1917 cells. Fluorescence microscopy images of: 11A) Cyborg E. coli MG1655 cells, and 11B) Cyborg E. coli Nissle 1917 cells. (n=3 independent experiments). See Methods Section M2. Scalebar=5 mm.

FIG. 12A-C: Metabolic activity of Cyborg E. coli MG1655 & Nissle 1917 cells. A&B) Metabolic activity of 12A) Wild Type and Cyborg E. coli MG1655 cells, and 12B) Wild Type and Cyborg E. coli Nissle 1917 cells right after hydrogelation. See Methods Section M4. Error bars=SD (n=4 independent experiments). 12C) Multi-day tracking of the metabolic activity of Wild Type and Cyborg E. coli Nissle 1917. The maximum fluorescence intensity of each daily assay is shown.

FIG. 13A-B: Tracking of Cyborg E. coli BL21 (DE3) Cells expressing mOrange. 13A) OD600 tracking of Wild Type and Cyborg E. coli BL21 (DE3) Cells induced and uninduced to express mOrange. 13B) Fluorescence tracking of Wild Type and Cyborg E. coli BL21 (DE3) Cells induced and uninduced to express mOrange. See Methods Section M11.Error bars=SD (n=4 independent experiments).

FIG. 14A-C: Phenotypic characterization of Cyborg E. coli BL21 (DE3) Cells. 14A) Cyborg E. coli BL21 (DE3) pLysS, pIURKL-mOrange do not express mOrange proteins when they are incubated in media without nutrients (1XPBS 1.5% LMTA). In the presence of nutrients and under the same experimental conditions, Cyborg Cells express mOrange (FIG. 2B). See Methods Section M5. Scalebar=5 μm. 14B) Cyborg E. coli BL21 (DE3) Cells divide without restriction in the absence of carbenicillin. (Scalebar=5 μm). 14C) Cyborg E. coli BL21 (DE3) Cells expressing mOrange undergo cellular lysis in the presence of carbenicillin. (Scalebar=5 μm).

FIG. 15A-B: Proteomic Data Analysis. 15A) Sample Loading Normalization. See Methods Section 12. 15B) Volcano plots showing the individual proteins being up (blue dots) and down (red dots) regulated in E. coli BL21 (DE3) Cells treated with only UV (UV-Treated), co-incubated with hydrogelation buffer (HG-Treated), and hydrogelated (Cyborg Cells). Individual proteins compared to the mean value in Wild Type Cells.

FIG. 16: Analysis of Individual Functional Protein Groups. Volcano plots showing individual proteins that are up (blue dots) and down (red dots) regulated in E. coli BL21 (DE3) Cells (UV-Treated, HG-Treated, and Cyborg Cells) across 17 different functional protein groups. Top panels: UV-treated. Middle panels: HG-treated. Bottom panels: Cyborg Cells.

FIG. 17A-B: Cyborg Cells remain functional with D-Cycloserine treatment. 17A) Cyborg Cells incubated in a media containing D-Cycloserine express mOrange in response to IPTG induction. 17B) Wild Type Cells incubated in a media containing D-Cycloserine are lysed. Scalebar=5 mm.

FIG. 18A-B: Cyborg E. coli BL21 (DE3) Cells remain functional at high pH. Fluorescence microscopy images showing replicates of Wild Type and Cyborg Cells incubated in a rich media with different pH values (7-9). 18A) Incubation of Wild Type and Cyborg Cells in media with pH 7. 18B) Incubation of Wild Type and Cyborg Cells in media with pH 8. 18C) Incubation of Wild Type and Cyborg Cells in media with pH 9. n=3 independent experiments. Scalebar=5 mm.

FIG. 19A-B: SDS PAGE analysis of E. coli expressing Invasin. 19A) Analysis of the soluble fraction of 6 different clones. 19) Analysis of the insoluble fraction of 6 different clones showing invasin expression. Arrows show the location of the expected invasin (top), and mOrange bands in both gels.

FIG. 20A-C: Analysis of the invasion of Cyborg Cells into cancer cells. 20A&B) Additional fake colorized microscope images of SH-SY5Y incubated with Cyborg E. coli BL21 (DE3) Cells expressing invasin and mOrange (A), and mOrange only (B) Scalebar=10 mm. 20C) Images used for training observers in the blind counting test. Positive-invasion. Negative-no invasion. See Methods Section M14.

### DEFINITIONS

Practicing this invention utilizes routine techniques in the field of molecular biology. Basic texts disclosing the general methods of use in this invention include Sambrook and Russell, Molecular Cloning. A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides can be performed using any art-recognized strategy, e.g., native acrylamide gel electrophoresis or anion-exchange high performance liquid chromatography (HPLC), e.g., as described in Pearson and Reanier, J. Chrom. 255: 137-149 (1983).

As used herein, the following terms have the meanings ascribed to them unless specified otherwise.

The terms “a.” “an,” or “the” as used herein not only include aspects with one member, but also include aspects with more than one member. For instance, the singular forms “a.” “an,” and “the” include plural referents unless the context clearly dictates otherwise. Thus, for example, reference to “a cell” includes a plurality of such cells, and so forth.

The term “nucleic acid” or “polynucleotide” refers to deoxyribonucleic acids (DNA) or ribonucleic acids (RNA) and polymers thereof in either single-or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); and Rossolini et al., Mol. Cell. Probes 8: 91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter. In some embodiments, the promoter is a prokaryotic promoter. Typical prokaryotic promoters include elements such as short sequences at the -10 and -35 positions upstream from the transcription start site, such as a Pribnow box at the -10 position typically consisting of the six nucleotides TATAAT, and a sequence at the -35 position, e.g., the six nucleotides TTGACA. In some embodiments, the promoter is a eukaryotic promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide.” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of a RNA and/or a nucleic acid sequence encoding a protein. In some embodiments, the term refers to the production of a transcriptional and/or translational product encoded by a gene (or a portion thereof. The level of expression of a DNA molecule in a cell may be assessed on the basis of either the amount of corresponding mRNA that is present within the cell or the amount of protein encoded by that DNA produced by the cell.

### DETAILED DESCRIPTION OF THE INVENTION

The inventors have discovered how to generate a synthetic polymer network inside cells, rendering them incapable of dividing while retaining cellular activity. The disclosure provides, for example, cells that comprise the synthetic polymer networks, as well as methods for making and using such cells.

It is believed that the synthetic polymer networks can be introduced into any cell using the methods described herein. Thus, for example, the cells can be prokaryotic or eukaryotic cells. Exemplary prokaryotic cells can be for example gram-negative or gram-positive cells. The methods described in the Examples are used to introduce hydrogel networks into E. coli but can be readily used for other gram negative prokaryotic cells, including but not limited to those of the genera Escherichia, Proteus, Enterobacter, Klebsiella, Citrobacter, Yersinia, Shigella, Pseudomonas, and Salmonella. It is expected that the intracellular hydrogelation protocol will work in gram-positive bacteria as well, optionally with minor modifications of the infusion process including the use of chemical enhancers such as CaCl₂ or the use of two or more freeze and thaw cycles to infuse hydrogel components inside gram-positive cells instead of a single cycle as demonstrated in the Example for gram-negative bacteria.

In some embodiments, the cells are eukaryotic cells. Exemplary eukaryotic cells can include but are not limited to fungal, plant, or mammalian cells. Exemplary fungal cells can include for example yeast cells, which can include but is not limited to Saccharomyces cerevisiae. Exemplary mammalian cells can include for example human cells. Exemplary human cells can include but are not limited to for example HeLa, HEK293, and SH-SY5Y cells.

The cells comprising the introduced hydrogel polymer networks lose their ability to replicate (e.g., to divide). Nevertheless, the cells retain metabolic and other cellular activities for a time period (e.g., a period of days), and thus can be used for one or more of their cellular activities without expansion of the cell population. For example, essentially all cellular functions aside from replication will continue to function. Exemplary cellular activity can include but is not limited to, transcription and translation, enzymatic functions, motility, REDOX reactions, homeostasis, and active response to stimuli in the environment. In some embodiments, the cells comprising the hydrogel polymer network will gain additional abilities or functions. For example as descried in the Examples, the cells can gain the ability to survive exposure to oxidative stress (e.g., H₂O₂ (10% w/w, 3M) for 3 hours at 37° C.).

Moreover, in some embodiments, the cell can be engineered to express one or more heterologous polynucleotide. In some embodiments, the expressed polynucleotide is an RNA that encodes a polypeptide. Thus, in some embodiments, the cells comprise one or more heterologous polynucleotide (e.g., DNA) that is integrated into its genome or provided on a plasmid or other extrachromosomal vector. In some embodiments, the polynucleotide is operably linked to an endogenous or heterologous promoter that controls expression of the polynucleotide. The promoter can, in some embodiments, be constitutive or inducible. When under an inducible promoter, expression of the gene product can be controlled by exposure of the cells to an agent that induces expression from the inducible promoter. In some embodiments, the cells produce or express one or more anti-cancer molecules, including but not limited to those described in, e.g., Briolay et al., Mol. Cancer. 20, 1-24 (2021). In some embodiments, the anti-cancer molecule is a RNA-guided nuclease, which can include but is not limited to Cas9, optionally with one or more guide RNAs.

The hydrogel polymeric network in the cells can be composed of different hydrogel

components as desired. The hydrogel will be formed from monomer subunits that are polymerized once introduced into the cell as discussed in more detail below. The monomer subunits can comprise for example poly-and/or mono-saccharides and/or proteins. As exemplified in the Examples, the hydrogel can be composed of poly (ethylene glycol) monomers of any of a variety of lengths. In other embodiments, the hydrogel can comprise for example, poly (dimethyl siloxane) (PDMS), poly (ethylene oxide) (PEO), poly (vinyl alcohol) (PVA), or poly (propylene fumarate) (PPF). Polymeric networks in cells can also be formed from components such as, but not limited to, alginate, guanosine mono phosphate (GMP), cyclodextrins (CD), fibrin, collagen, polypeptides, decellularized extracellular matrix, and nucleic acids. A discussion of hydrogels can be found in, e.g., Ahmed, Journal of Advanced Research, Volume 6, Issue 2, March 2015, Pages 105-121; Drury J L. Mooney D J. 2003 Biomaterials. 24 (24); 4337-4351; and Tibbitt M W, Anseth K S. 2009. Biotechnol. Bioeng. 103 (4); 655-663.

The hydrogel monomers can be substituted at one or more side chains, e.g., with a side chain moiety or otherwise conjugated to other molecules, providing further functionality or reactivity of the polymer in the cell. For example, some or all of the monomers of the polymers can be conjugated to, for example, metals, nucleic acids, nanoparticles, peptides, and drugs. Generally, conjugation to the monomers will occur before the monomers are introduced into the cells but in some embodiments, conjugation can occur after the monomer subunits are introduced into the cells. In some embodiments, the conjugation is carried out using activated monomers that can be conjugated to peptides, drugs, enzymes, etc. An activated monomer is a monomer that has been modified with a reactive (electrophilic) group. For example, PEG or other monomers can be modified with aryl chloride residues, reactive acyl groups, or modified with alkylating reagents or optionally can be purchased commercially.

Cells having an internal hydrogel polymer can be generated by providing cells, introducing hydrogel monomeric subunits and depending on the monomeric subunit, a polymerization inducer (an initiator of polymerization), into the cells and then polymerizing the monomeric subunits in the cells to form the polymer network in the cells. As noted above, any cell can be used as a starting material. Generally, the cells will be cultured such that the cells are actively dividing. As an example, for bacterial cells, they can be cultured so that the cells are in exponential growth phase. Other cells may not have an exponential growth phase, but can nevertheless be exposed to new culture and nutrients such that the cells are in a phase of cell division.

The monomer subunits and polymerization inducer can be introduced into the cells as desired. In some embodiments, for example, the cells can be exposed to a freeze/thaw cycle to increase the cells' ability to uptake the monomeric subunits and polymerization inducer. The freezing will be selected to avoid excessive cell death while improving the entry of the monomer subunits. For example, the cells can be “flash’ frozen, e.g., immersed in a liquid below zero degrees C., e.g., at −80 C, for less than five minutes, e.g., 1-3 minutes. In some embodiments, one or more cryoprotectant is incubated with the cells during the freeze/thaw cycle to protect the cells from the process. Exemplary cryoprotectants include, but are not limited to, dimethyl sulfoxide (DMSO) or glycerol. In other embodiments, the monomer subunits can be introduced into the cell by chemical or electrical (e.g., electroporation)-based methods, or methods including but not limited to, e.g., osmotic shock, sonoporation, electroporation, laser, surfactant-based permeabilization, or shearing. Once the monomers are introduced into the cells, the cells can be washed to remove excess monomers on the exterior of the cells.

The concentration of monomers introduced into the cells can be selected for optimum results and can vary depending on the monomer and resulting polymers used. In some embodiments, the concentration of monomers is 1-30% (w/w), e.g., 1-20%, 1-10%, 3-8% or 4-6%.

In some embodiments, the monomers are poly ethylene glycol (PEG) molecules. As one example, the PEG monomers can be of an average weight of 500-2000 daltons. In some embodiments, the PEG monomers comprise end moieties to assist in polymerization. For example, in some embodiments, the PEG is a diacrylate, meaning both ends comprise acrylate moieties.

In some embodiments, the monomers are sodium alginate, a non-toxic, biocompatible, and biodegradable polysaccharide. The formation of hydrogels from alginate can occur by interactions of the anionic alginates with multivalent inorganic cations through a typical ionotropic gelation method. Exemplary non-limiting divalent cations can include, for example, calcium or magnesium. The monomer and the crosslinker (divalent cation) can be introduced into the cell as desired. In some embodiments, they are introduced together or separately by freezing and thawing, osmotic shock, sonoporation, electroporation, laser, surfactant-based permeabilization, or shearing.

In some embodiments, the monomers are guanosine. In these embodiments, hydrogelation can occur as self-assembly, e.g., to form a guanosine mono phosphate (GMP) hydrogel. In some embodiments, the hydrogel can be stabilized using hydrazides, aldehydes, or cations such as but not limited to K+, which can also be introduced into the cells. The monomer and the stabilizer can be introduced for example by freezing and thawing, osmotic shock, sonoporation, electroporation, laser, surfactant-based permeabilization, or shearing.

In some embodiments, the monomers are cyclodextrin (CD), i.e., a cyclic oligosaccharide of glucopyranoside units linked through α-1,4 glycosidic bonds. Once introduced into cells, a CD polymer can be formed. In some embodiments, the polymer is formed via chemical cross-linking, for example by free-radical polymerization cross-linking-based methods; nucleophilic addition/substitution-based methods; cross-linking methods based on ‘click’ reactions and/or incorporation of CDs through post-gelation attachment. The CD monomer and any crosslinkers can be introduced, for example, by freezing and thawing, osmotic shock, sonoporation, electroporation, laser, surfactant-based permeabilization, or shearing.

In some embodiments, the monomers are fibrinogen (e.g., a glycoprotein 45 nm in length). Once introduced into cells, fibrin polymerization can be initiated by the action of the proteolytic enzyme, thrombin, which can also be introduced into the cells. The monomer and polymerizing enzyme (e.g., thrombin) can be introduced, for example, by freezing and thawing, osmotic shock, sonoporation, electroporation, laser, surfactant-based permeabilization, or shearing.

In some embodiments, the monomers are collagen. For example, in some embodiments, the collagen is type I collagen subunits (triple-helical protein formed of 67-nm periodic polypeptide chains with a total molecular weight near 300 kDa). Polymerization can be achieved, for example with a mixture of temperature, pH, and ionic strength. In some embodiments, the monomers can be introduced by freezing and thawing, osmotic shock, sonoporation, electroporation, laser, surfactant-based permeabilization, or shearing. In some embodiments, crosslinking factors is controlled, for example, independently of permeation. For example, one can change temperature, ionic strength, and pH of the cell culture medium to induce cross-linking without needing to introduce external factors into the cell.

In some embodiments such as extracellular matrix (ECM), the monomers variate

and their precise composition can depend on the tissue that it is derived from. For example, the composition is a mixture of collagen, glycosaminoglycans, proteoglycans, and ECM proteins. Hydrogel formation can be induced in cells by collagen-based self-assembly, which can also be regulated, for example, by a mixture of temperature, pH, and ionic strength. The ECM components can be introduced by freezing and thawing, osmotic shock, sonoporation, electroporation, laser, surfactant-based permeabilization, or shearing. In some embodiments, crosslinking factors are controlled independently of permeation. For example, one can change temperature, ionic strength, and pH to induce cross-linking without needing to introduce external factors into the cell.

In some embodiments, the monomers are nucleic acids or nucleotides. Nucleic acid hydrogels can be formed within the cells. In some embodiments, different classes of nucleic acids (e.g., DNA molecules) with different properties and shapes (e.g., X-shaped, Y-shaped or T-shaped DNA molecules). In some embodiments, the nucleic acids are chemically or enzymatically cross-linked to form a hydrogel in the cells. In some embodiments, the nucleic acids and, if included, crosslinking enzymes (e.g., ligases used to polymerize different shapes of DNA), can be introduced, for example, by freezing and thawing, osmotic shock, sonoporation, electroporation, laser, surfactant-based permeabilization, or shearing.

In some embodiments, the monomers are polydimethylsiloxane-isocyanatomethyl-3,5,5-trimethylcyclohexyl isocyanate-2-hydroxyethylmethacry late (PDMS-IPDI-HEMA) and acrylamide. In some embodiments, these components can be polymerized in the cells, for example, by micellar copolymerization (compartmentalization of PDMS-IPDI-HEMA inside SDS or other detergent micelles). PDMS-IPDI-HEMA micelles and acrylamide can be introduced to cells, for example by freezing and thawing, osmotic shock, sonoporation, electroporation, laser, surfactant-based permeabilization, or shearing.

In some embodiments, the monomers are poly (ethylene oxide) (PEO). The PEO monomers can be any of a variety of weights as desired. In some embodiments, the lengths of the PEO monomers differ. In some embodiments, the PEO monomers can be of average length of 10000-10 million daltons weights (e.g., 35,000:900,000; or 5,000,000 Da). In some embodiments, the PEO monomers are of different molecular weights (e.g., 35,000; 900,000; and/or 5,000,000 Da). Polymerization of the monomers, once introduced into the cells, can be achieved for example, by y-irradiation or photo-crosslinking using UV light. PEO monomers can be introduced into cells, for example, by freezing and thawing, osmotic shock, sonoporation, electroporation, laser, surfactant-based permeabilization, or shearing.

In some embodiments, the monomers are poly (vinyl alcohol) (PVA). Following introduction into the cells, polymerization can be carried out, for example, by low-temperature crystallization. PVA can be introduced into cells, for example, by osmotic shock, sonoporation, electroporation, laser, surfactant-based permeabilization, or shearing.

In some embodiments, the monomers are poly (propylene fumarate) (PPF). In some embodiments, the monomers are a mixture of PPF and PEG. For example, in some embodiments, a 1:1 w/w mixture of PPF and PEG can be introduced into cells and then formed into a hydrogel with low cytotoxicity. In some embodiments, a polymerization inducer is also introduced into the cell. Exemplary polymerization inducer can include, for example, a benzoyl peroxide initiator mixed with a vinyl monomer, N-vinyl pyrrolidinone. The reaction can be further accelerated with, for example, N,N-dimethyl-p-toluidine. These components can be introduced into the cells to initiate polymerization. Monomers and other components described above can be introduced into the cells, optionally in gradual steps, by freezing and thawing, osmotic shock, sonoporation, electroporation, laser, surfactant-based permeabilization, or shearing.

Depending on the monomer and polymerization inducer employed, the polymerization inducer can be introduced into the cells with the monomers or in a separate step. For efficiency, it can be helpful to introduce both at the same time. However, in circumstances in which mixture of the monomer and inducer causes significant polymerization before the components can be introduced into the cell together, it can be advantageous to introduce each component separately into the cells. The inducer used will depend on the cross-reaction chemistry involved in linking the monomers in the cell. In some embodiments, the inducer can be selected from, for example, 2-hydroxyl-4′-(2-hydroxyethoxy)-2-methylpropiophenone, 2-hydroxy-4′-(2-hydroxyethoxy)-2-methylpropiophenone (Irgacure 2959), Eosin-Y, and lithium phenyl-2,4,6-tri-methylbenzoylphosphinate.

Once the monomer subunits and polymerization inducer are introduced into the cells, polymerization can be induced. In embodiments in which the inducer is a photo-inducer, the appropriate wavelength and intensity of light can be exposed to the cells for sufficient time to polymerize the monomer subunits thereby forming a polymer network within the cell. In other embodiments, the inducer can be activated by temperature, or other environmental stimuli, or in some embodiments, simply by being in proximity to the monomer subunits.

In some embodiments, following polymerization, the cells are exposed to a replication-specific toxin and/or antibiotics that target the replication of cells. This will kill cells that remain capable of replication, thereby enriching cells that comprise the hydrogel polymer and that are no longer capable of replication. Exemplary toxins or antibiotics can include, but are not limited to, carbenicillin, which kills replicated bacterial cells.

The resulting cell population will comprise cells having an internal polymeric network that prevents the cells from dividing but that nevertheless retain other biological activity. These cells can then be used in a large variety of biological assays, utilizing the native biological mechanisms of the cell or one or more heterologous activity in the cell resulting from, for example, expression of one or more heterologous polynucleotide in the cells.

In some embodiments, one can modulate one or more metabolic pathways in the cells. This can be achieved to maximize the funneling of resources towards metabolic routes where cellular resources are necessary therefore maximizing the function of the cells and increasing their life span. In some embodiments, for example, one can inhibit Dihydrofolate reductase (DHFR) and the folate pathway by contacting the cells, for example, by using Trimethoprim. In some embodiments, for example, one can inhibit fatty-acid and lipid synthesis, for example using ACHN-975, Cerulenin, or Platencin. In some embodiments, for example, one can inhibit RNA polymerase and RNA synthesis, for example, using Rifampicin and/or Norfloxacin. In some embodiments, for example, one can inhibit ribosomes and protein synthesis, for example, using Kanamycin. In some embodiments, for example, one can inhibit protein degradation, for example, using Z-LY-CMK and Bortezomib.

In some embodiments, a biological activity of the cells can be measured, e.g., in response to exposure to one or more agents, expressed gene product, or environmental change. In some embodiments, the cells can be exposed to one or more cell-permeable probes to measure a biological activity. Exemplary cell-permeable probes include but are not limited to, resazurin-based PrestoBlue™ (ThermoFisher Scientific). In other embodiments, one can measure the ATP/ADP ratio of the cells using a protein biosensor. An exemplary protein biosensor can include but is not limited to, Perceval (see, e.g., Tantama, et al., Nat Commun. 2013; 4: 2550). In yet other embodiments, the cells can be measured for proteomic and metabolomic changes in response to exposure to one or more agents, expressed gene products, or environmental experience.

In some embodiments, the cells comprising the polymeric network can be put in contact with a population of different cells. This can occur in vitro, ex vivo, or in vivo. In some embodiments, for example, the cells comprising the polymeric network express one or more heterologous proteins that are harmful to or promote a response in a second cell population. In some embodiments, for example, the cells comprising the polymeric network express Invasin (Wong et al., Curr. Opin. Microbiol. 8, 4-9 (2005)), a protein that assists the cells comprising the polymeric network to penetrate other cells. In some embodiments, the targeted cells are tumors or other cancer cells, for example in or from a human. In some embodiments, the cells comprising the polymeric network can be used as probiotics.

### EXAMPLE

The following example is meant to exemplify, but not limit, the invention.

Here, we demonstrate the convergence of both living and artificial systems, creating semi-living cells with the engineering simplicity of synthetic materials and the complex functionalities of natural cells. We show hydrogel crosslinking inside bacterial cytoplasm under specific conditions can create non-replicating yet metabolically active entities, which are herein termed Cyborg Cells. Cyborg Cells preserve genetic material integrity, fluid and functional cell membrane interfaces, and active metabolic pathways. The non-dividing property of Cyborg Cells renders them incapable of contaminating the ecosystems like living synthetic cells. Furthermore, Cyborg Cells gain new abilities in resisting stressors that would otherwise kill their unmodified counterparts. Cyborg Cells can be created using different bacterial strains and modified with existing synthetic biology parts readily. Lastly, we demonstrate the ability of Cyborg Cells to invade cancer cells in vitro. This study showcases how a hybrid approach in the intersection between materials and synthetic biology can result in a cellular platform with non-natural, engineered, and modular functions, adding to the growing efforts in live cell engineering with synthetic materials (L. Schoonen, J. C. M. Van Hest, Adv. Mater. 28, 1109-1128 (2016); Dai et al., Nat. Chem. Biol. 15, 1017-1024 (2019); Sigmund et al., Nat. Commun. 9 (2018), doi: 10.1038/s41467-018-04227-3; Lau et al., Nat. Commun. 9 (2018), doi: 10.1038/s41467-018-03768-x; Justus et al., Sci. Robot. 4, 1-19 (2019); Huang et al., Nat. Chem. Biol. 15, 34-41 (2019); Lin et al., Nat. Commun. 10, 1-11 (2019); Tang et al., Nat. Chem. Biol. 17, 724-731 (2021); Liu et al., Proc. Natl. Acad. Sci. U. S. A. 114, 2200-2205 (2017)).

### Results

### Engineering Cyborg Cells Through Intracellular Hydrogelation

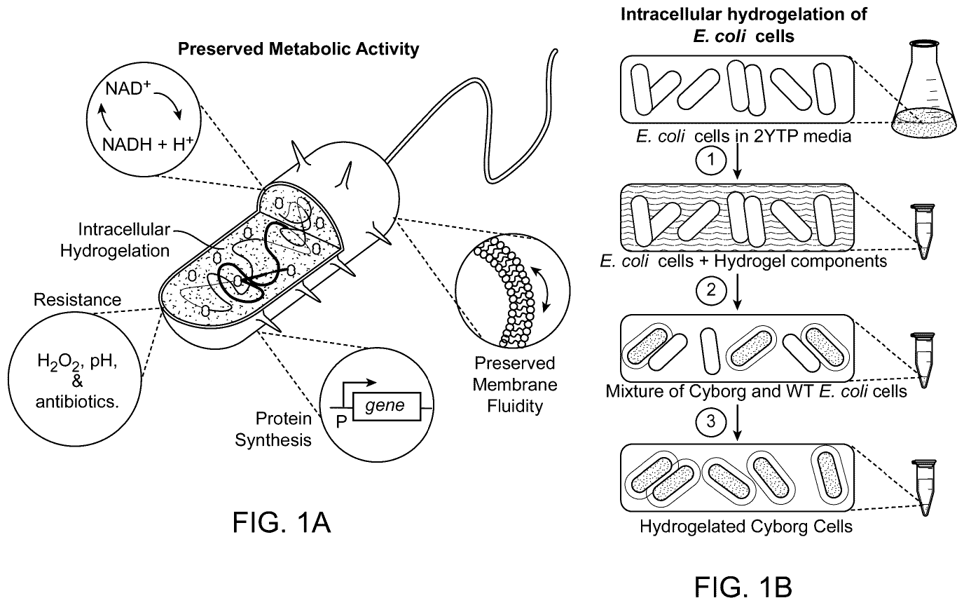

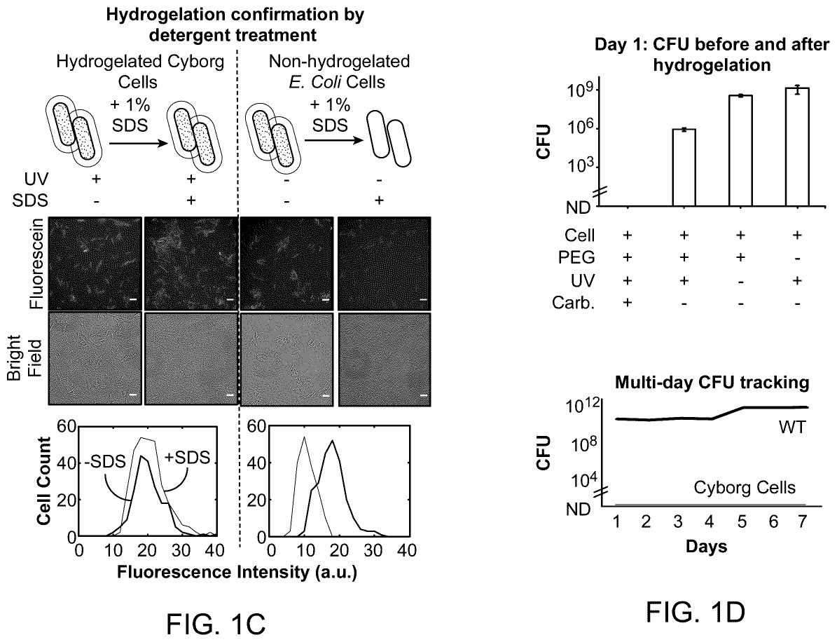

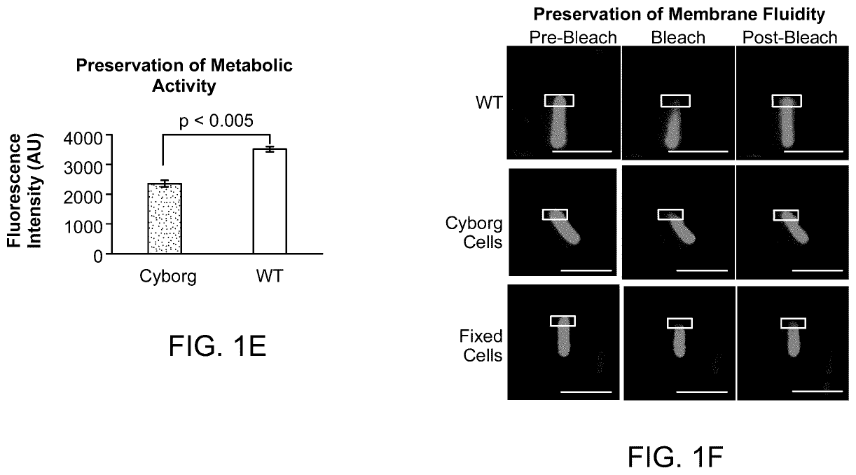

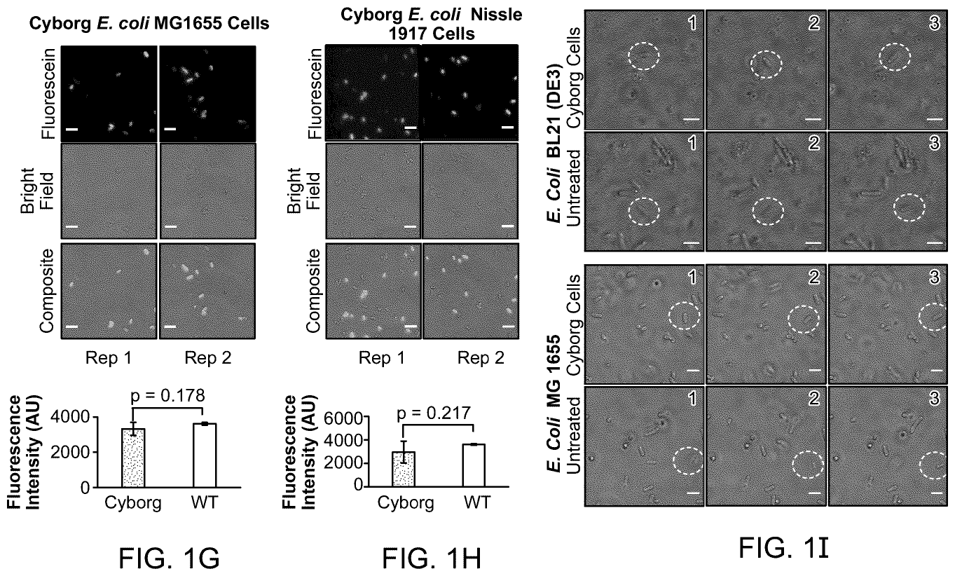

We envisioned the creation of a bio-micromachine chassis with similar capabilities as natural bacterial cells, but with enhanced characteristics provided by their modification with a synthetic material. The chassis would preserve key characteristics of living cells, including cellular metabolism, protein synthesis, membrane fluidity, and functionality of membrane proteins. In addition, the chassis would ideally gain new non-native functions and would lack the capacity to propagate (FIG. 1A).

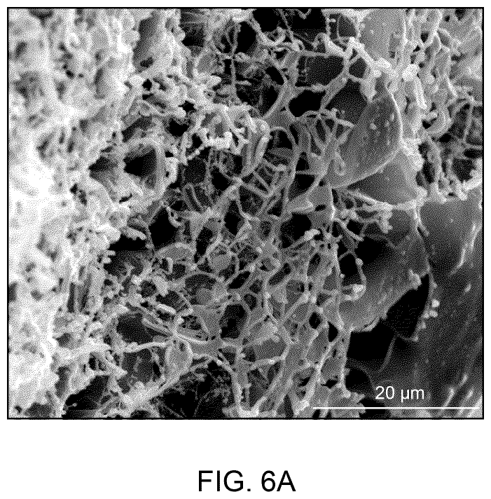

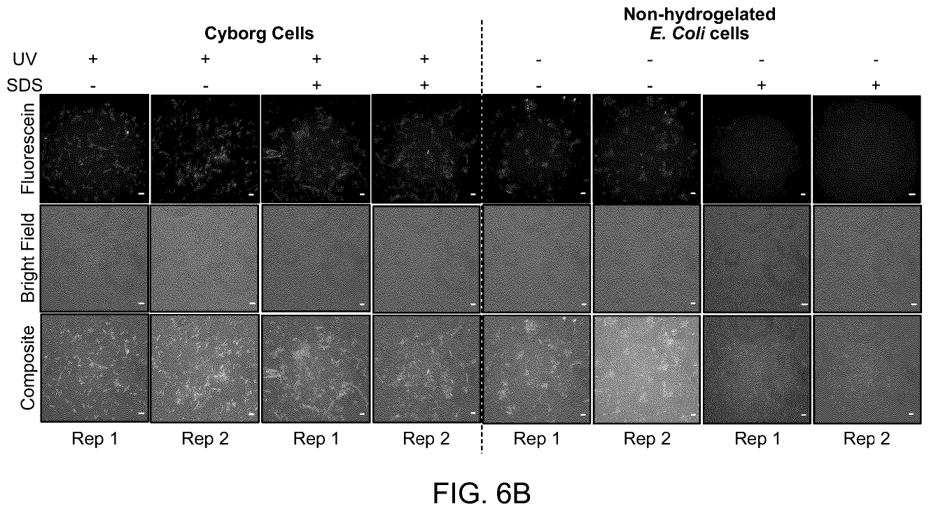

To create a chassis with the desired traits, we infused bacteria with a synthetic hydrogel with low biological reactivity (Park et al., Nat. Mater. 11, 895-905 (2012); Lin et al.,Nat. Commun. 10, 1-11 (2019); Underhill et al., Biomaterials. 28, 256-270 (2007)). The selected hydrogel chemistry consists of poly (ethylene glycol) diacrylate monomer (PEG-DA; Mn 700) and 2-hydroxyl-4′-(2-hydroxyethoxy)-2-methylpropiophenone as the photoinitiator. We also incorporated fluorescein O′O-diacrylate as a fluorescent dye to check the permeation of the hydrogelation components into the bacteria and the success of intracellular hydrogelation (FIGS. 1B&C, Methods Sections M2 & M3). The infusion of bacterial cells with the hydrogel components was conducted using a single freeze-thaw cycle. After successful infusion of the hydrogel components, the bacterial cells were washed to remove extracellular hydrogel components and cell debris. Crosslinking of the intracellular PEG monomers was triggered using ultraviolet light (UV-A), which was purposefully chosen to minimize absorption by DNA and other biological components. A 5% hydrogel density was chosen for the intracellular hydrogelation as the particular gel density gives rise to a highly porous scaffold upon crosslinking (Methods Sections M6, FIG. 6A). After ultraviolet light irradiation, the resulting bacterial cells were incubated in a rich media (37° C., 250 rpm) and treated with carbenicillin to kill replicating cells. The carbenicillin treatment eliminated bacterial cells that were not successfully hydrogelated, therefore yielding a population of Cyborg cells unable to divide (FIG. 1B). To confirm hydrogelation, we treated both hydrogelated (+UV) and non-hydrogelated cells (−UV) infused with hydrogel with 1% SDS.

an ionic detergent commonly used for the rapid disruption of biological membranes. Hydrogelated cells retained their green fluorescence when compared to non-hydrogelated cells infused with hydrogel (FIG. 1C, FIG. 6B). These results are consistent with standard validation assays in the field showing the successful crosslinking of PEG hydrogels. Thus, we confirmed that hydrogel is successfully formed inside bacteria and that hydrogelated bacteria can be identified using the dye-based fluorescence imaging.

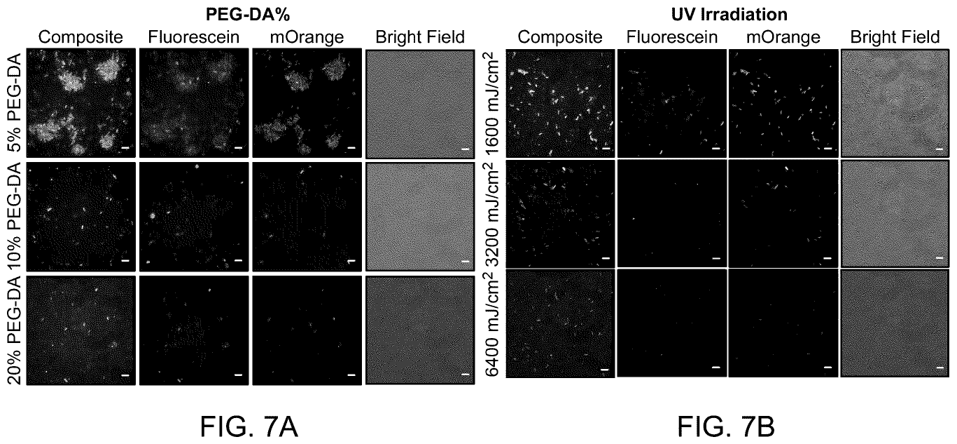

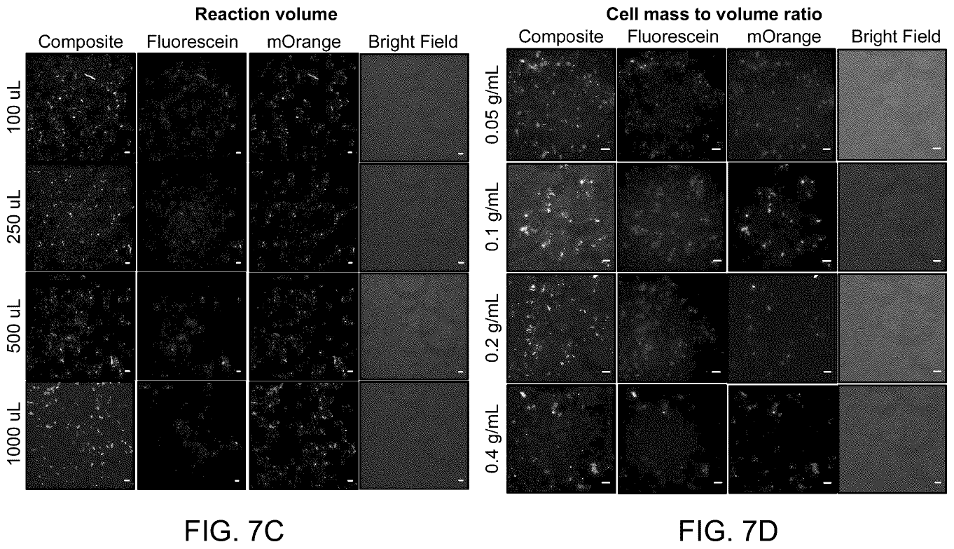

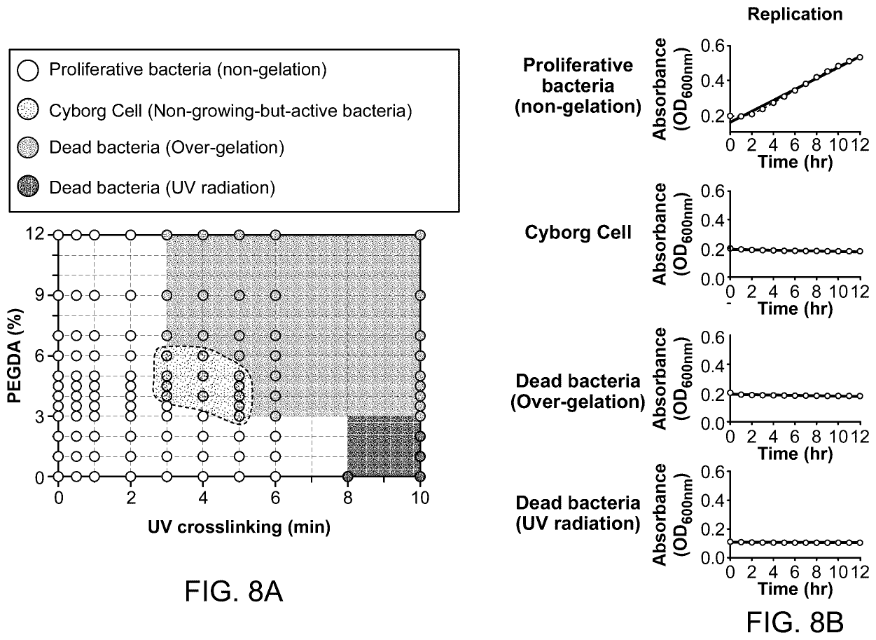

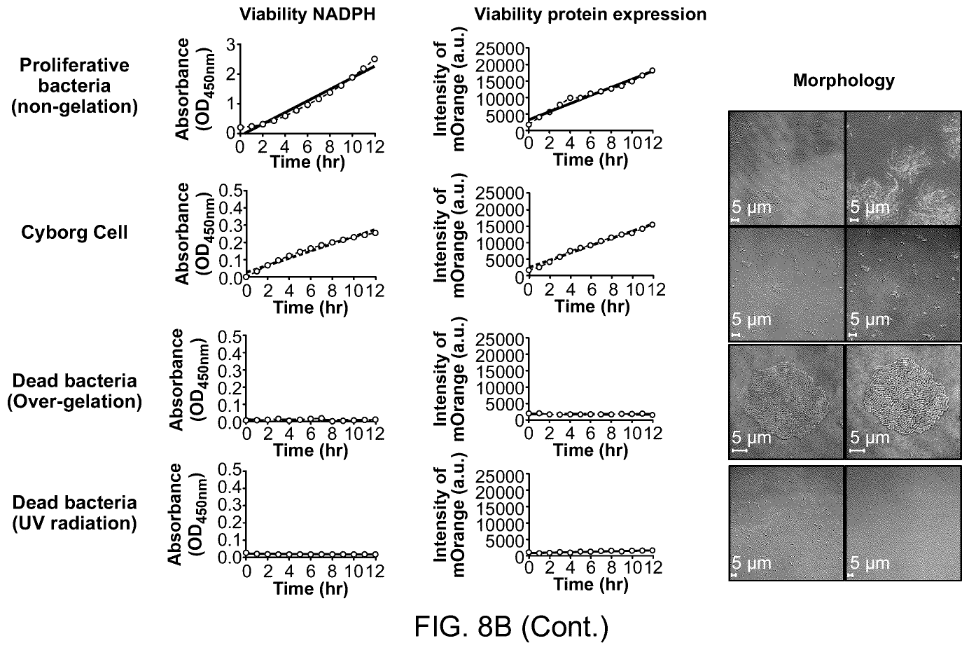

Next, we optimized the generation of Cyborg Cells by modifying different parameters, including UV irradiation duration, membrane permeation protocol, and concentrations of crosslinking and culturing reagents (i.e., PEG, photoactivator, and antibiotics). For each perturbation, we characterized the phenotype of the resulting hydrogelated cells to map the key parameters required to produce the non-growing-but-active Cyborg Cells (FIGS. 7 & 8). We note that Cyborg Cells could only be generated within a specific hydrogelation window, and non-optimal conditions resulted in dead cells or non-hydrogelated cells. To ensure the robustness of our results, we replicated the hydrogelation of bacteria in two different labs at UC Davis (California, USA) and Academia Sinica (Taipei, Taiwan). Repeated generation of the Cyborg Cells suggests that cytoplasmic hydrogel of a particular density allows for biomolecular movements responsible for certain metabolism actions but deprives bacteria of their capability to grow and divide.

Using our optimized protocol, we examined the replication ability of the hydrogelated bacteria using a Colony Forming Unit (CFU) assay (Methods Section M8) and compared the CFU counts obtained from Cyborg Cells, cells incubated with hydrogel components but not treated with UV, and cells treated only with UV (FIG. 1D). Before carbenicillin treatment, the CFU of the Cyborg Cell population was 3-log 10 fold lower than our non-hydrogelated controls. After carbenicillin treatment, the CFU of Cyborg Cells decreased to a non-detectable level. The carbenicillin treatment served to purify the Cyborg Cells population, and Cyborg Cells although nonreplicable, remained active and intact after the treatment (FIG. 1E-I). Further experiments demonstrated that over six days of CFU tracking. Cyborg Cells showed no detectable CFUs. Meanwhile, Wild Type cells grew to high density levels with daily dilutions (FIG. 1D, bottom panel) (for all subsequent results, “Wild Type” denotes the original non-hydrogelated bacteria). The results confirmed that we can produce a population of intracellular hydrogelated bacteria unable to divide.

### The Cyborg Cells Maintain the Active Features of Natural Cells

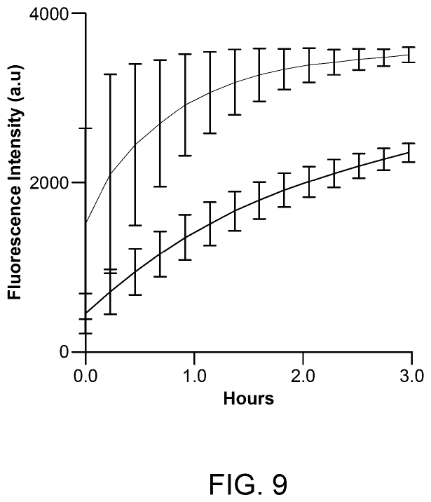

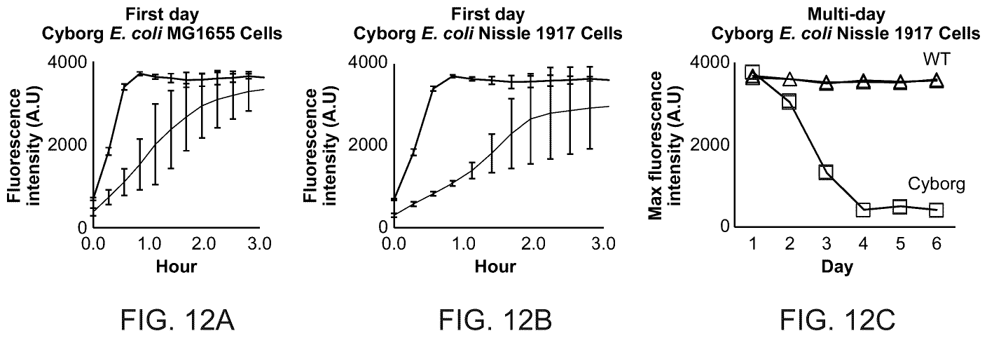

Furthermore, we characterized the preservation of essential cellular activities after hydrogelation. We assessed the metabolic activity of the Cyborg Cells using a cell viability reagent (PrestoBlue™, ThermoFisher Scientific) that reports the reducing power of living cells (Methods Section M4), which is commonly associated with the state of cellular metabolism (Xu et al., J. Pharmacol. Toxicol. Methods. 71, 1-7 (2015)). Our Cyborg Cells exhibited ˜70% of the maximum metabolic activity on the first day, without showing any measurable growth (FIG. 1E and FIG. 9). Further experiments showed that our Cyborg Cells preserve quantifiable metabolic activity for up to three days (FIG. 12C), equivalent to ˜150 division cycles of natural bacteria. Living cells preserved constant metabolic activity for the duration of the experiment as expected for a cell population that divides continuously in a rich media. Our results show that the Cyborg Cells preserve reducing power, suggesting the continual functioning of major metabolic pathways.

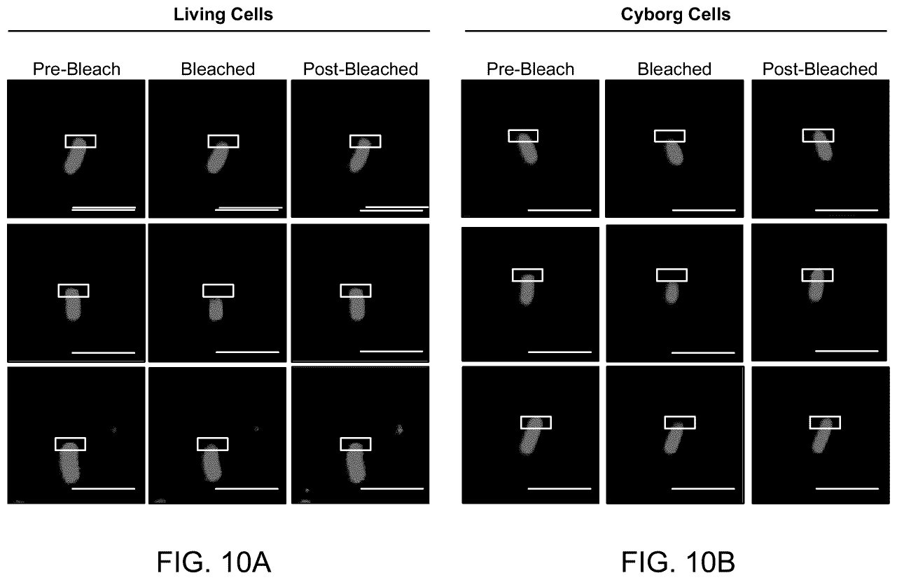

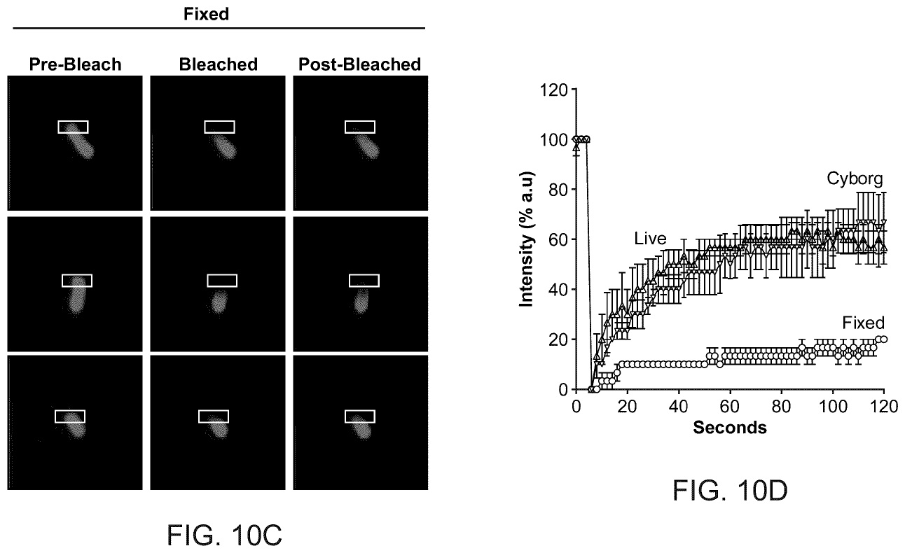

Another key cellular characteristic that we aimed to preserve in our Cyborg Cells is membrane fluidity, a parameter associated with the correct function and viability of bacterial cells (Gohrbandt et al., EMBO J., 1-21 (2022)). We performed fluorescence recovery after photobleaching (FRAP) assays using a lipophilic DiD dye to assess the state of the lipid membrane in our Cyborg Cells (Methods Section M7). Using this method, we analyzed the membrane fluidity of three different E. coli BL21 (DE3) populations: untreated cells, Cyborg Cells, and fixed cells (FIG. 1F). Cyborg Cells and untreated cells showed similar recovery halftimes after photobleaching, while fixed cells showed significantly higher recovery halftimes (FIG. 1F, FIG. 10).

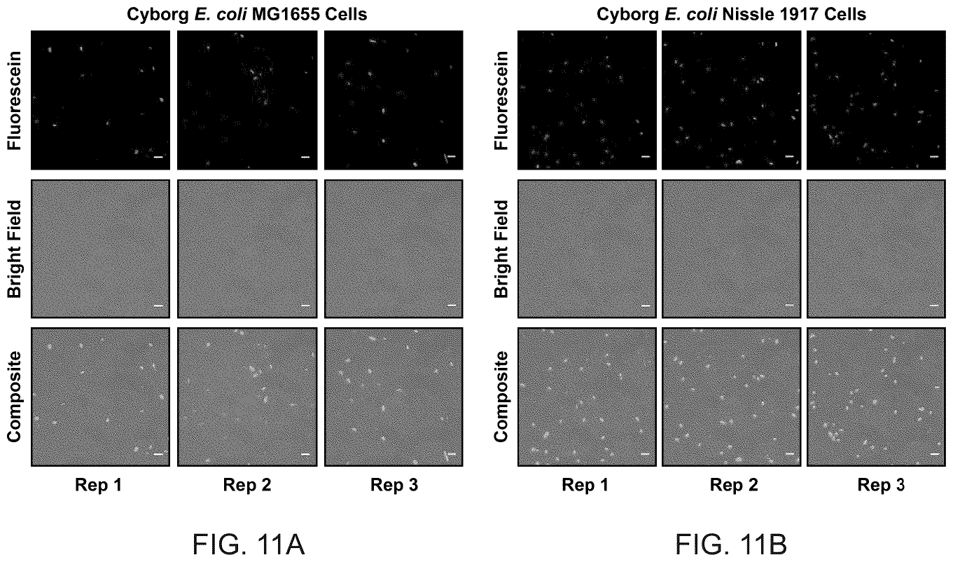

In contrast to genetic approaches for cell-chassis engineering, hydrogel-mediated Cyborg Cell generation could be readily applied to different E. coli strains without considering the genetic context. Hence, we tested the intracellular hydrogelation protocol on two different strains with different genotypes and lineages from E. coli BL21 (DE3); E. coli MG1655 and the probiotic strain Nissle 1917. Consistent with our earlier results, hydrogel was readily established inside Cyborg MG1655 and Nissle 1917 Cells under the light-mediated radical polymerization protocol (FIG. 1G&H, top panels. FIG. 11). Furthermore, both strains showed similar metabolic activity to Wild Type cells (FIG. 1G&H, bottom panels, FIG. 12), confirming that our hydrogelation protocol can produce Cyborg E. coli Cells regardless of the genetic context of the tested strains. Furthermore, Cyborg E. coli MG1655 and Cyborg E. coli BL21 (DE3) showed similar motility to the Wild Type cells (FIG. 1I, Methods Section M9). Altogether, these results demonstrate that our approach creates hydrogelated bacteria that cannot divide, while preserving membrane fluidity, motility, and metabolic activity.

### Inducible Protein Expression and Proteomic Changes in Cyborg Cells

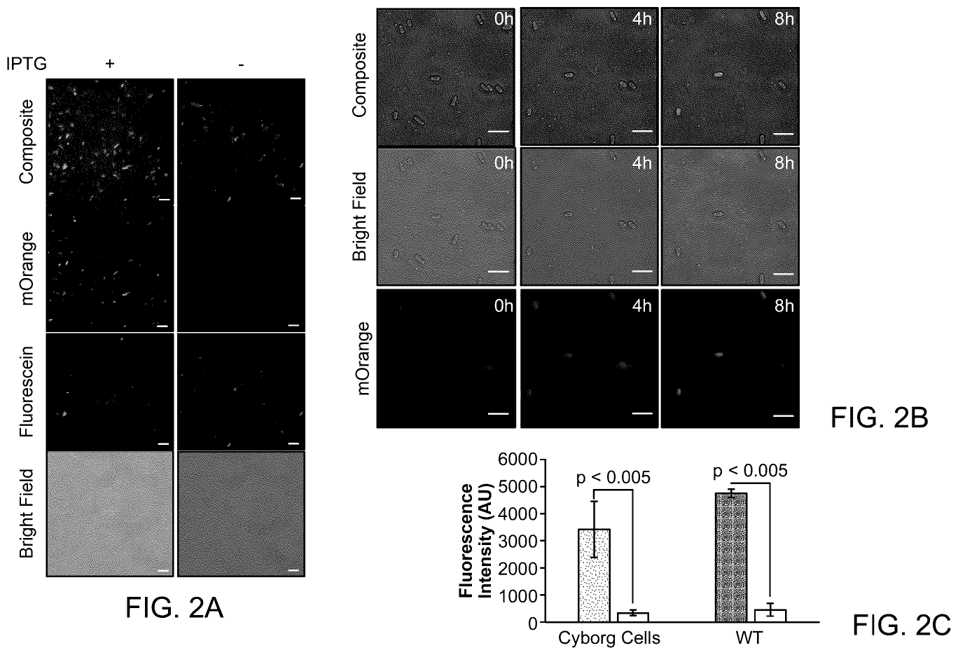

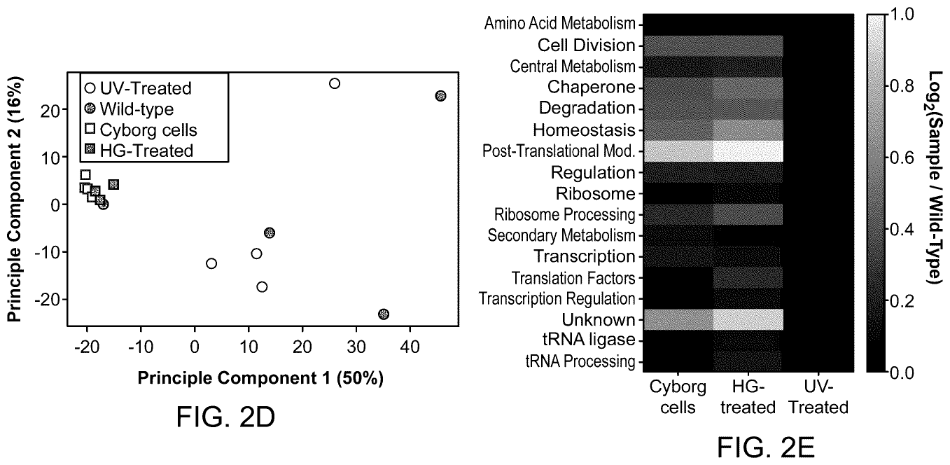

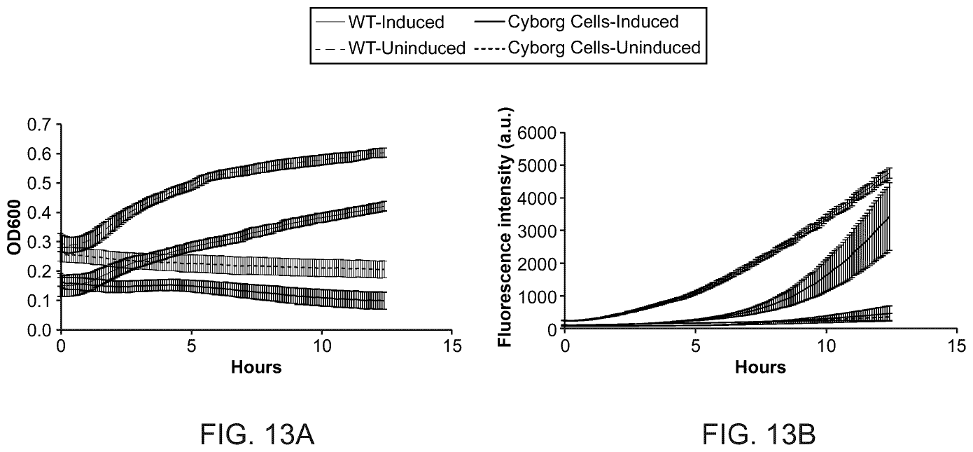

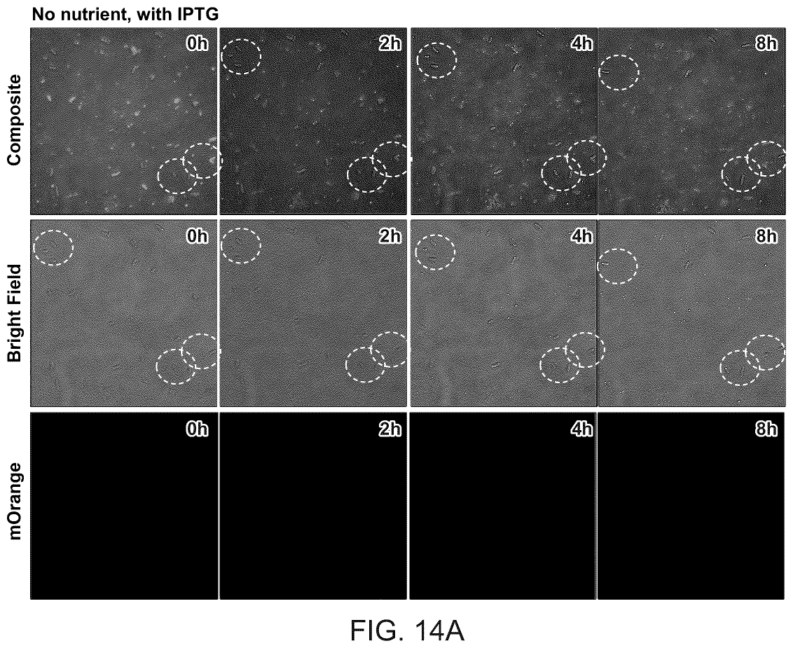

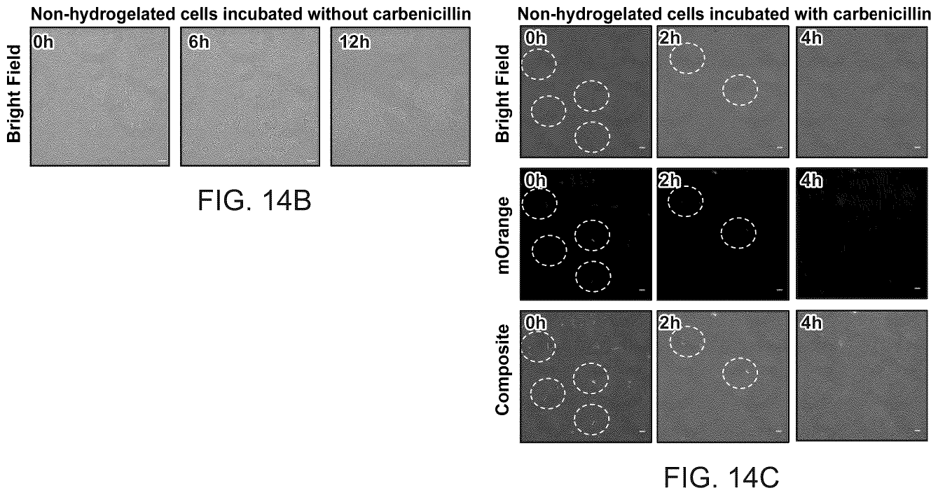

We next examined the use of inducible genetic circuits inside Cyborg Cells. First, we created Cyborg Cells using the strain E. coli BL21 (DE3) containing a plasmid encoding the fluorescent reporter mOrange under the control of the PT7-lacO hybrid promoter (Methods Section M1). We used this strain to assess if Cyborg Cells retained protein expression capabilities. Specifically, we used fluorescence microscopy (Methods Section M5) and a plate reader (Methods Section M11) to assess if our Cyborg Cells were capable of inducible protein expression (FIG. 2A-C). Fluorescence microscopy images showed that Cyborg Cells expressed mOrange in response to IPTG induction (FIG. 2A). Moreover, our experiments showed that Cyborg Cells expressed 70% of the total mOrange produced by Wild Type cells despite exhibiting no cellular growth (FIG. 2C. FIG. 13). On the contrary. Wild Type cells showed an expected increase in cell density (FIG. 13). Additionally, we used fluorescence microscopy to track the real-time expression of fluorescent reporter mOrange to further assess for bacterial replication (FIG. 2B, Methods Section M5). The results reaffirm the Cyborg Cells' non-replicative nature and demonstrate continued protein expression following chemical induction. Additional experiments also demonstrated that our Cyborg Cells express proteins only in the presence of nutrients (FIG. 14A), suggesting that nutrient uptake and metabolism are essential for Cyborg Cells activity. On the other hand. non-hydrogelated cells incubated under the same conditions replicate in the absence of carbenicillin (FIG. 14B) or die in the presence of carbenicillin (FIG. 14C).

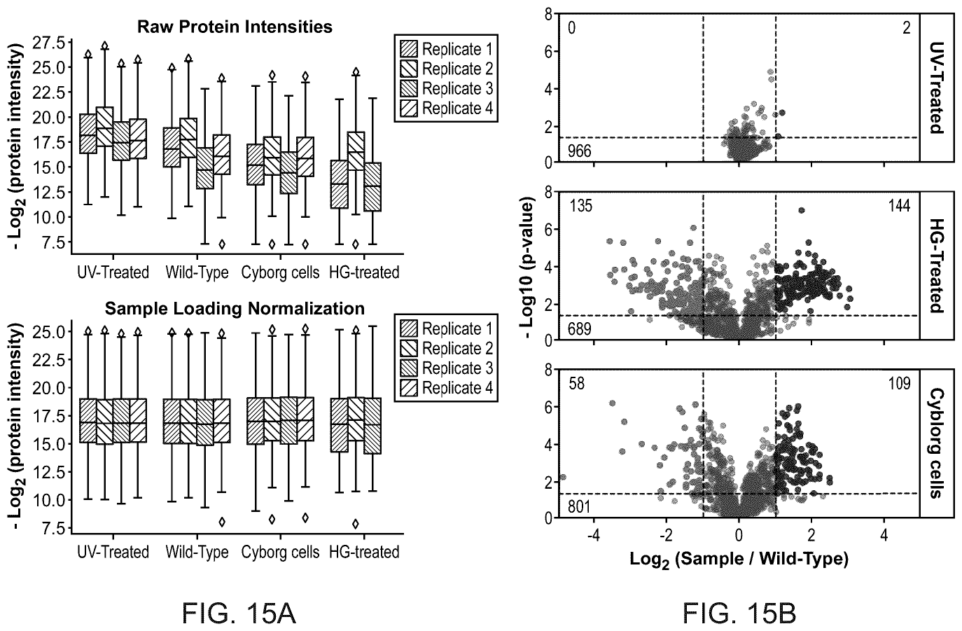

Furthermore, we analyzed the protein composition of our Cyborg Cells through mass spectrometry to identify key proteome changes in response to hydrogelation (FIGS. 2D&E. FIGS. 15 &16. Methods Section M12). The proteomic content of Cyborg Cells was compared to cell extracts produced from untreated controls (Wild Type Cells), bacterial cells treated with UV (UV-Treated), and bacterial cells incubated with hydrogel without light-activated crosslinking (HG-treated). We used the strain E. coli BL21 (DE3) to produce the proteomic extracts of the Cyborg Cells and the controls mentioned above. After standard hydrogelation (Methods Section M2) and processing of the different controls, we performed the digestion, labeling, and tandem mass tag (TMT) mass spectrometry in quadruplicate (Methods Section M12). Mass spectrometry analysis revealed the levels of ˜900 proteins in all samples. After signal normalization (FIG. 15A. Methods Section M12), principal component analysis (PCA) shows clustering and separation of experimental conditions according to the type of sample (FIG. 2D). The data show close clustering between Cyborg and HG-treated cells, whereas Wild Type and UV-treated cells are clustered separately.

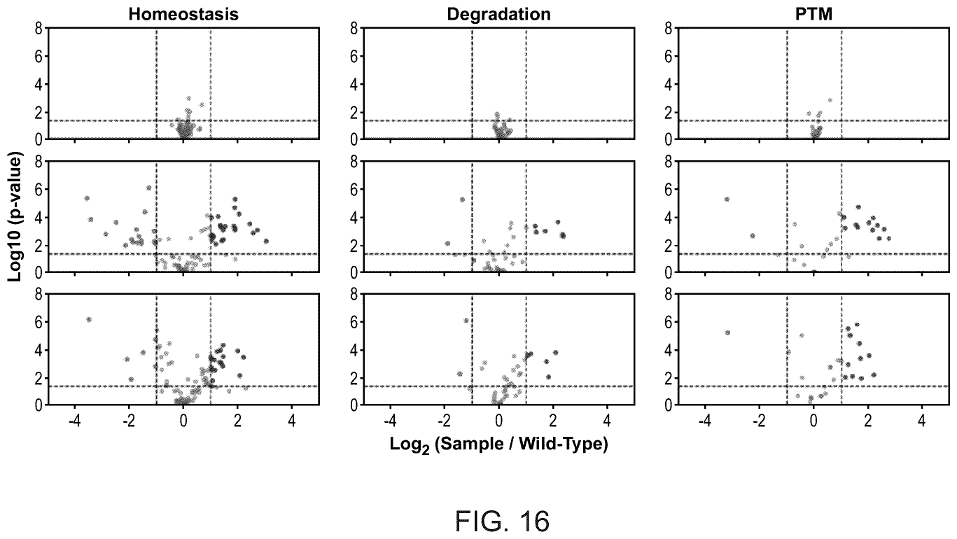

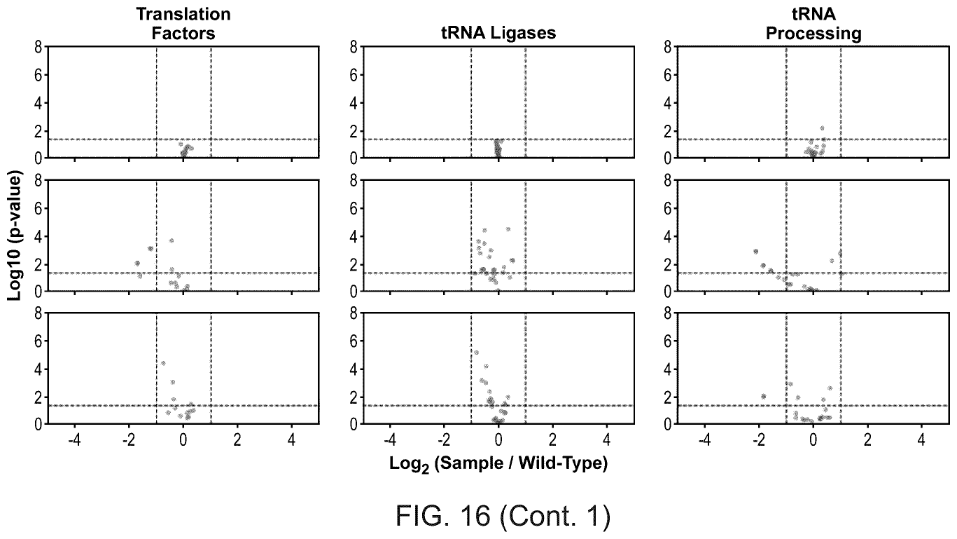

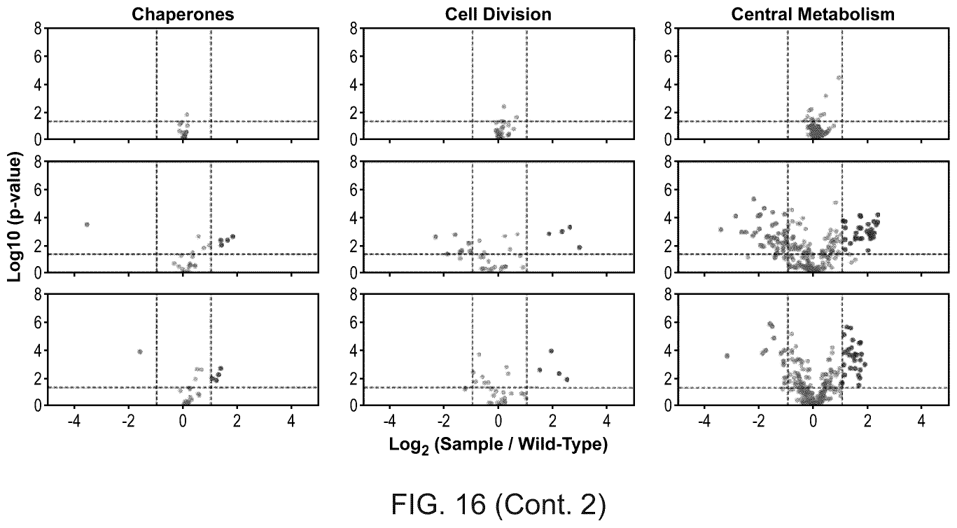

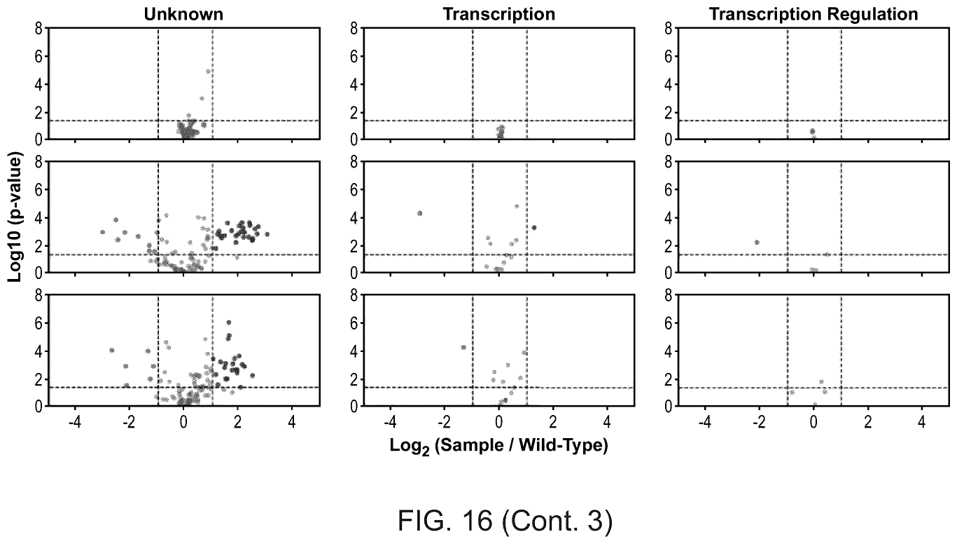

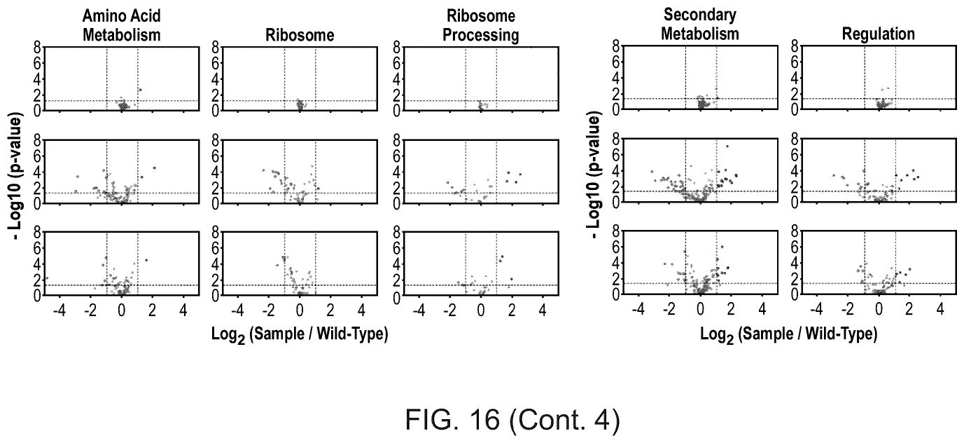

Moreover, we investigated the proteomic changes that are driving the different clustering of each condition. To do this, we plotted the fold change of each protein intensity using Wild Type as our reference and the p-value from a two-way t-test of that comparison (FIG. 15B). According to this analysis. UV-Treated cells mainly remained unchanged when compared to Wild Type. At the same time. HG-treated and Cyborg Cells showed statistically significant changes in 29% and 17% of the total number of proteins detected. respectively. To further characterize the proteomic changes, we classified each protein showing a significant change from our Wild Type control into functional groups (FIG. 16). Next, we compared the fold changes between the means of each protein to the Wild Type control and calculated the absolute value of the average of all fold changes in each functional category (FIG. 2E). Our analysis indicates that intracellular hydrogelation produces a significant upregulation of proteins involved in homeostasis, protein folding, degradation, central metabolism, and post-translational modification. In the homeostasis group, notable upregulated proteins include TolC family, RND efflux system, LPS assembly proteins, and Na+/H+ antiporter. In the post-translational modification group, some notable genes involved in protein assembly were upregulated: BamABCD, SecD, and LolBD. In the cell division group, FtsXNZ involved in the Z-ring formation was upregulated, but murAC involved in peptidoglycan biosynthesis was downregulated. The analysis shows that our Cyborg Cells have a different proteomic profile than their unmodified counterparts. The proteome profile suggests that the hydrogelation indirectly changes the composition of proteins involved in metabolism, protein synthesis, and protein assembly. Additionally, the proteome changes of the cell division functional group suggest that Cyborg Cells could be partially impeded to divide due to the downregulated biosynthesis of membrane components. Future work could investigate if the physical impact of intracellular hydrogelation on cellular volume occlusion, DNA replication, and cell segmentation also cause the inability of Cyborg Cells to divide.

### Cyborg Cells are Compatible With Synthetic Genetic Circuits

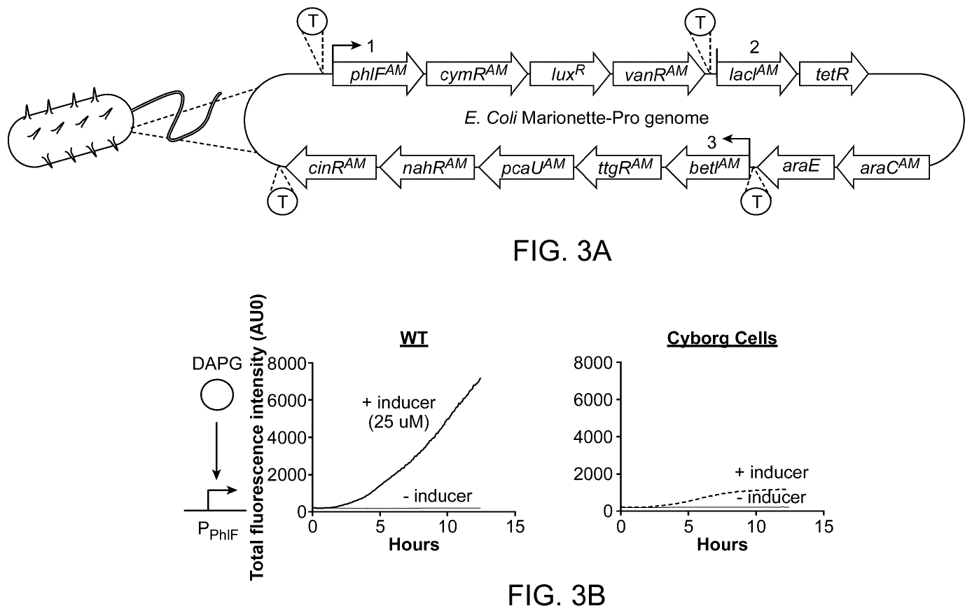

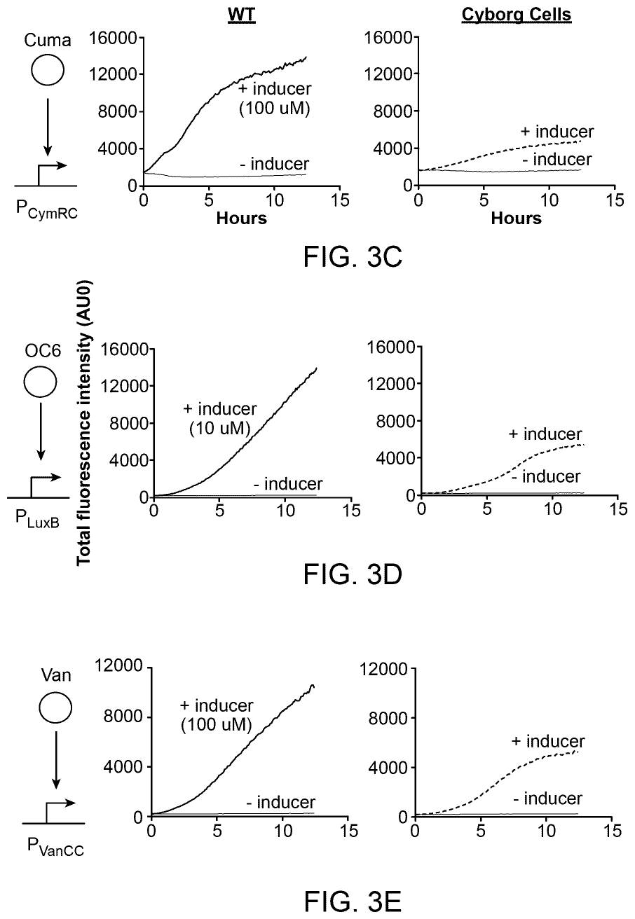

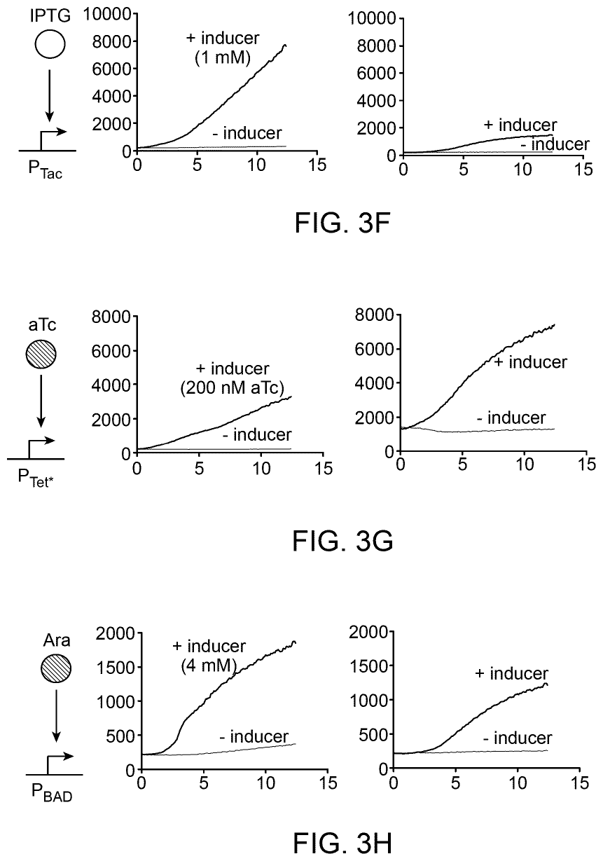

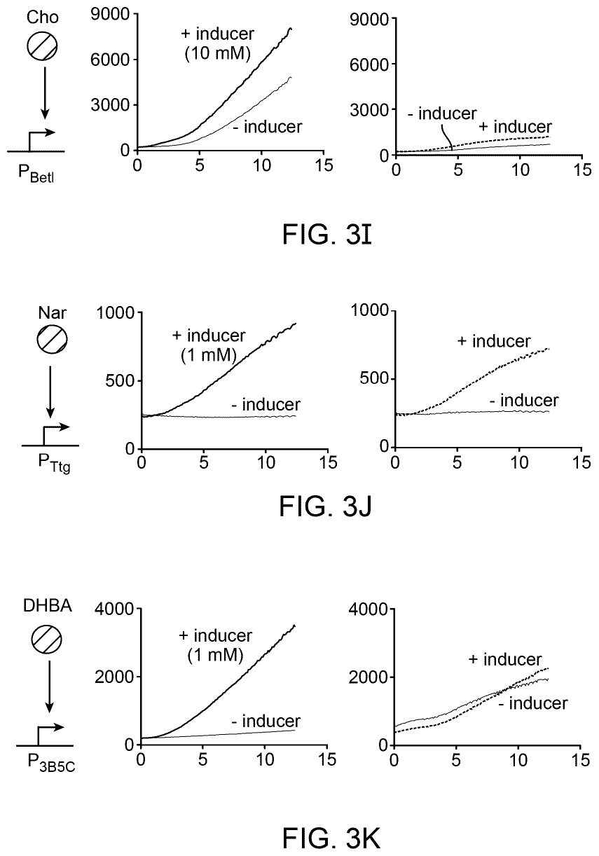

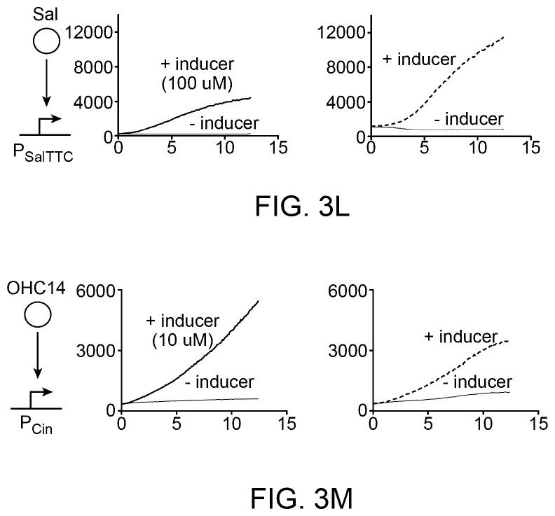

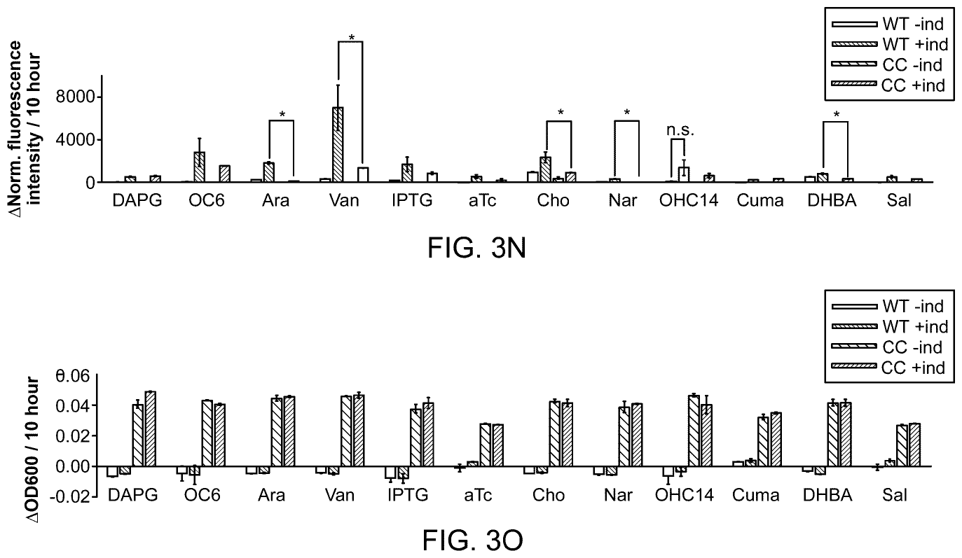

To further test the capabilities of the Cyborg Cells, we functionalized them using a library of small molecule sensors from the Marionette Sensor Collection (Meyer et al., Nat. Chem. Biol. 15, 196-204 (2019)). We examined if Cyborg Cells could be rapidly functionalized with different synthetic biology parts and if we could produce active and responsive Cyborg Cells using existing synthetic bacterial strains without further genetic changes. Using the plasmids and strains provided with the Marionette Sensor Collection, we created 12 Marionette Strains responsive to the small molecules 2,4-diacetylphophloroglucinol (DAPG), cuminic acid (Cuma), 3-oxohexanoyl-homoserine lactone (OC6), vanillic acid (Van), isopropyl β-d-1-thiogalactopyranoside (IPTG), anhydrotetracycline (aTc), 1-arabinose (Ara), choline chloride (Cho), naringenin (Nar), 3,4-dihydroxy benzoic acid (DHBA), sodium salicylate (Sal), and 3-hydroxytetradecanoyl-homoserine lactone (OHC14) (Methods Section M1). We then hydrogelated them (Methods Section M10) to create Cyborg Marionette Cells that respond to each of the 12 small molecule inducers (FIG. 3A). Immediately after hydrogelation, Cyborg Marionette Cells and Wild Type control cells were incubated with and without the appropriate inducer (FIG. 3B-M) and the expression kinetics and growth were tracked for 12 hours.

The 12 Cyborg Marionette Strains expressed the reporter YFP in response to the appropriate inducers and showed similar response compared to Wild Type controls (FIG. 3B-M). All Cyborg Marionette Strains showed increased expression of YFP over time in the presence of each inducer. For nearly all inducers (except aTc and Sal, FIG. 3G&L), Wild Type cells resulted in higher total fluorescence intensity of the cell population than Cyborg Cells. This result is expected due to the continuous growth of the Wild Type marionette cells contrary to the lack of growth by the Cyborg Marionette Cells (FIG. 3O). To approximate single-cell performance, we calculated the rate of YFP expression by normalizing fluorescence intensity values using OD600 data and then calculated the difference in the normalized fluorescence data at 0) and 10 hours (FIG. 3N). All Cyborg Marionette strains showed statistically significant differences (p-value<0).05) between uninduced and induced populations, except OHC14 (p-value=0.06). Cyborg Marionette Strains responded to Van, Ara, Cho, Nar, and DHBA at a higher rate than the corresponding Wild Type strains (p-value<0.05). All Cyborg Marionette strains showed negligible change in OD600, consistent with our earlier results about the non-growing property of Cyborg Cells (FIG. 3O). Using the Cyborg Marionette Strain responsive to 1-arabinose, we indirectly assessed the functionality of the arabinose-proton symporter encoded by the gene arak (Keasling et al., Microbiology. 147, 3241-3247 (2001)). Both Cyborg Marionette and Wild Type cells show a similar kinetic response to arabinose induction (FIG. 3H), suggesting a functional arabinose transporter in our Cyborg Cells, consistent with our earlier FRAP assay showing a fluid and functional cell membrane (FIG. 1F). Altogether, these results show that our Cyborg Cells can be functionalized with a diverse set of synthetic biology parts and readily generated from existing synthetic cells.

### Cyborg Cells GAIN NEW NON-NATIVE FUNCTIONALITIES

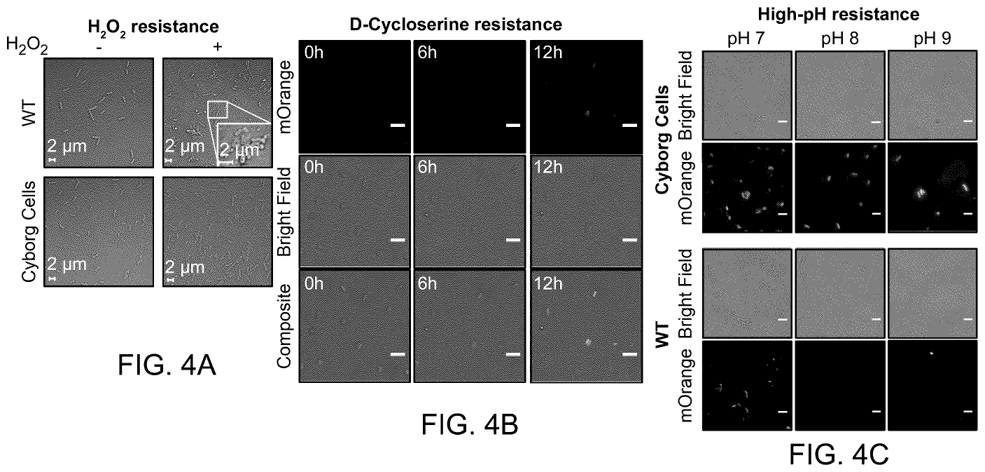

Thus far, we have demonstrated that our Cyborg Cells preserve key functions of living synthetic cells. Next, we tested if the synthetic hydrogel provides new capabilities to the Cyborg cells. We first examined if the Cyborg Bacteria could resist a hyper oxidative environment containing hydrogen peroxide (H₂O₂). H₂O₂ is an essential chemical component of host-defenses and degradation mechanisms in mammalian cells (Clifford et al., Mol. Cell. Biochem. 149, 143-149 (1982)). H₂O₂ kills bacterial cells by causing accumulation of irreversible oxidative damage to the membrane layers, cell wall, proteins, and DNA (Brudzynski et al., Front. Microbiol. 2, 1-9 (2011)). Using fluorescence microscopy, we tested if Cyborg E. coli (Migula) Castellani and Chalmers remained stable in the presence of a lethal concentration of H₂O₂ (10% w/w; 3M) for 3 hours at 37° C. and compared the results against Wild Type bacteria (FIG. 4A). E. coli Migula is commonly used for antimicrobial susceptibility and media testing. After incubation with H₂O₂, the morphology and shape of Cyborg Cells remained unchanged, while Wild Type bacteria underwent cell lysis, with fragments and debris observed in microscopy images. These results show that Cyborg Cells remain stable in hyper oxidative environments where Wild Type bacteria cannot survive, indicating that the synthetic hydrogel confers a degree of protection against damaging agents that otherwise will kill natural cells.

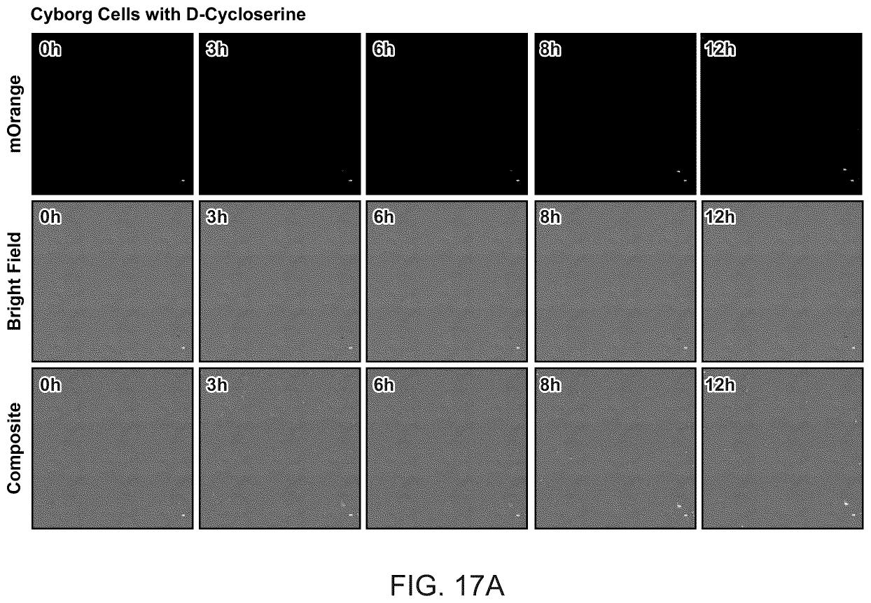

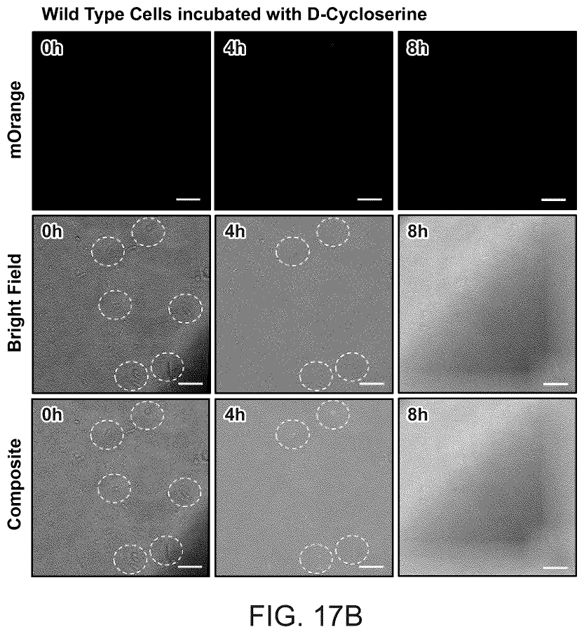

In addition, we tested the resistance of Cyborg Cells to cell wall-targeting antibiotics, D-Cycloserine (FIG. 4B, FIG. 17). D-Cycloserine is a potent antibiotic particularly effective against gram-negative bacteria commonly used to treat tuberculosis, and with a similar mechanism of action to the beta-lactam class of antibiotics. After hydrogelation, we treated Cyborg E. coli BL21 (DE3) bacteria with D-Cycloserine (200 ug uL⁻¹). We tracked both single-cell mOrange expression and cell survival for 12 h using fluorescence microscopy (Methods Section M5). Cyborg E. coli BL21 (DE3) Cells under D-Cycloserine treatment (FIG. 4B) continued to synthesize mOrange protein. In contrast, Wild Type cells lysed during the treatment (FIG. 17B).

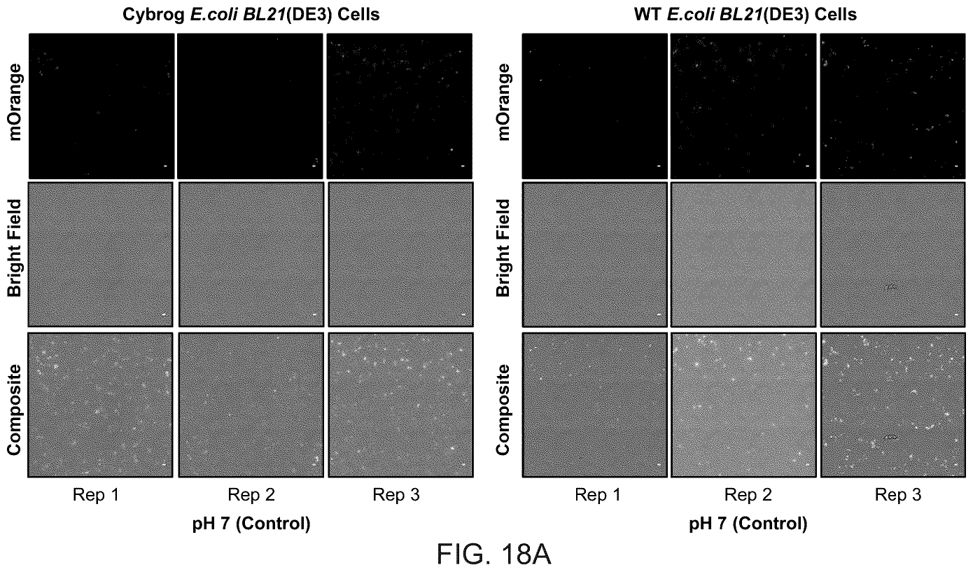

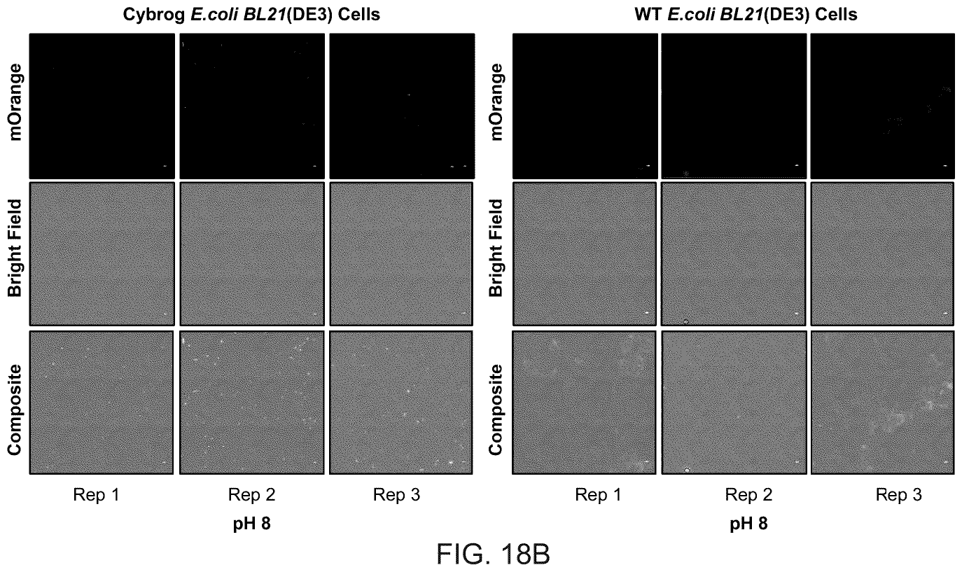

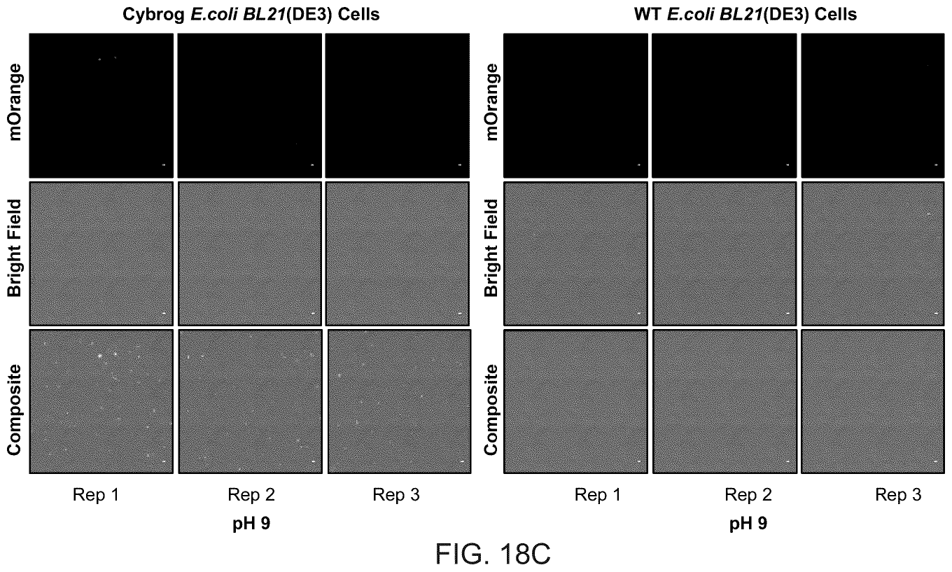

Next, we examined Cyborg Cells' behavior in media with high pH (>pH 7) (FIG. 4C). High salinity in both water and soils can cause environments with high pH where only alkaliphiles or alkali-tolerant bacteria can survive (Kulkarni et al., Microbial Diversity in the Genomic Era,pp. 239-263 (Elsevier, 2019)). Furthermore, organisms capable of functioning at elevated pH values are deemed essential for biomedical and industrial applications (Sarethy et al., J. Ind. Microbiol. Biotechnol. 38, 769-790 (2011); Padan et al., Biochim. Biophys. Acta-Biomembr. 1717, 67-88 (2005)). Our experiments show that Cyborg E. coli BL21 (DE3) Cells remained stable and expressed the fluorescent reporter mOrange in response to IPTG induction at pH 7-9 (FIG. 4C, FIG. 18). Non-hydrogelated Wild Type controls behaved similarly to Cyborg Cells at pH7. However, at pH 8, non-hydrogelated control cells formed filament cells and could not express mOrange. At pH 9, they lysed. These results show that hydrogelation confers Cyborg Cells new non-native capabilities in resisting the stressors, while Wild Type bacteria are killed by the stressors. Making Cyborg Cells that can survive in such conditions could enable new applications of synthetic cells for bioremediation and disease treatment.

### Cyborg Cells Can Invade Cancer Cells In Vitro

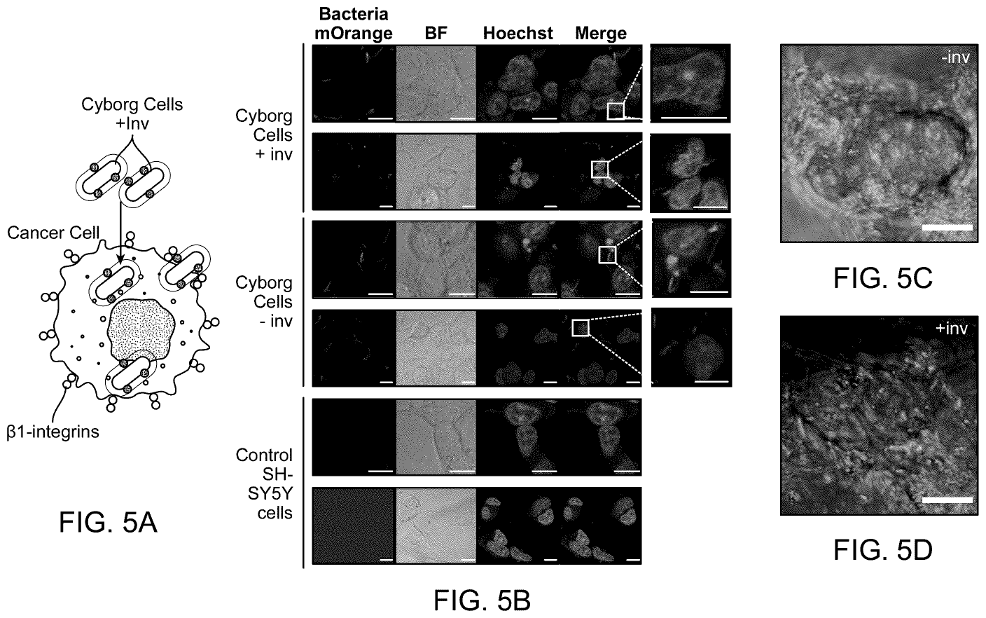

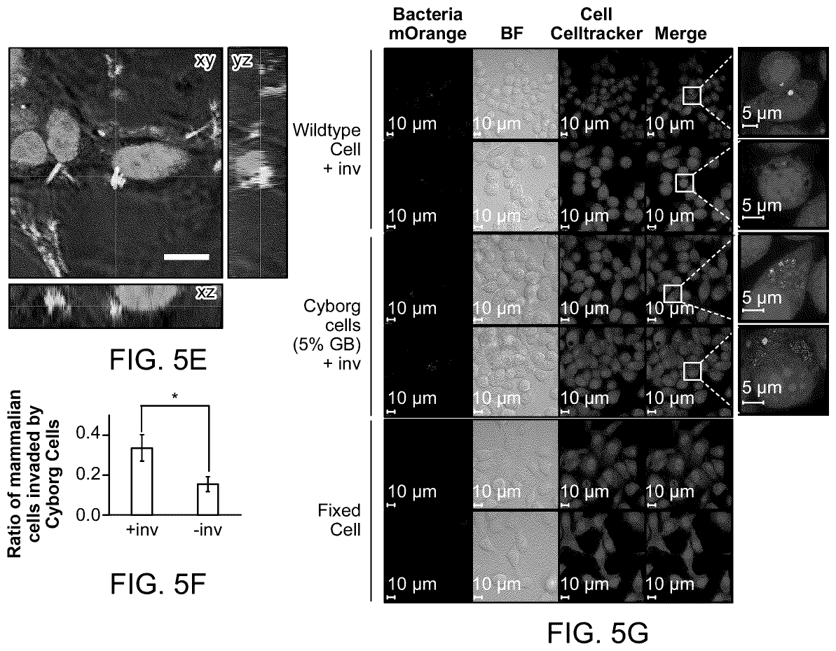

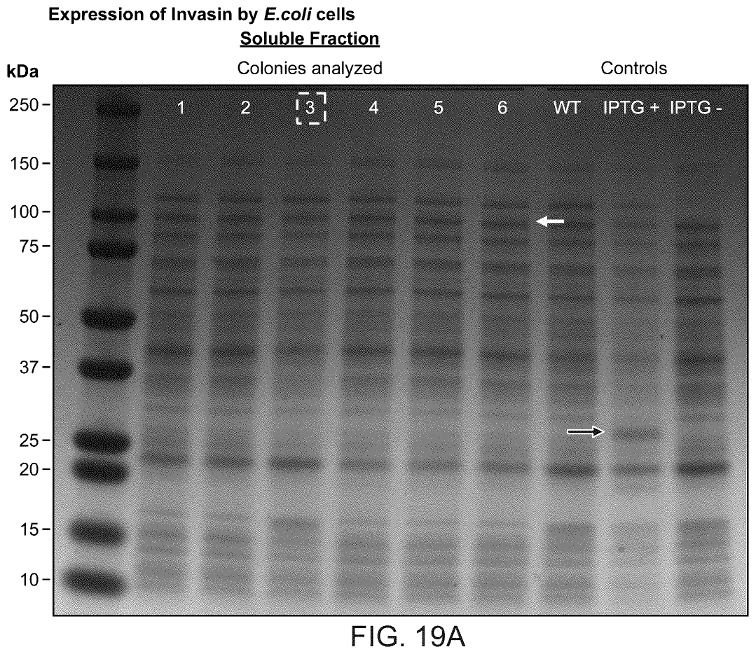

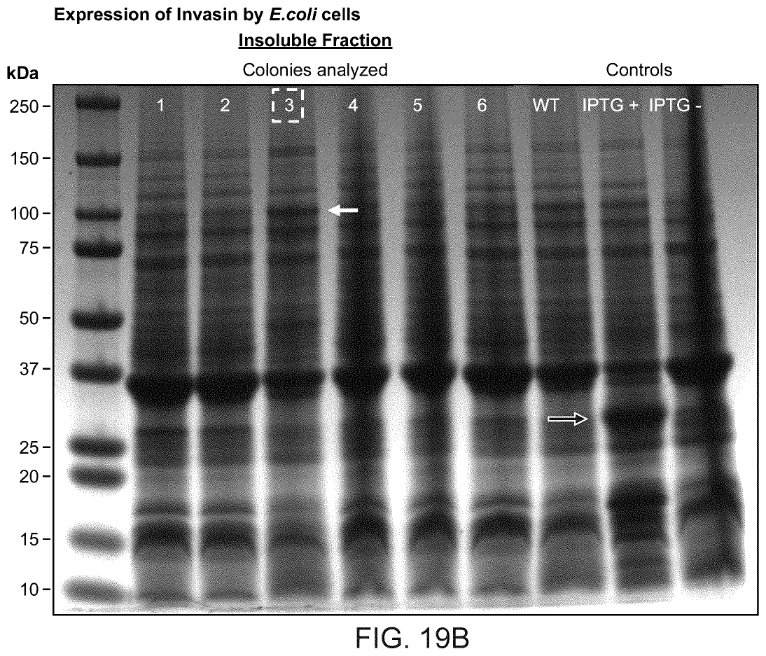

One of the main opportunities of synthetic biology in biomedicine is the delivery of therapeutics by tumor-invasive bacteria (Weber and Fussenegger, Nat. Rev. Genet. 13, 21-35 (2012)). Earlier experiments showed that our Cyborg Cells retain the functionality of their cell membranes and membrane proteins (FIG. 1F&2H). Based on the proven properties of Cyborg Cells, we engineered Cyborg Cells that could invade mammalian cells aided by the protein Invasin (FIG. 5A). Invasin is a single-gene protein from Yersinia pseudotuberculosis known to mediate adhesion and invasion of mammalian cells when expressed in E. coli (Anderson et al., J. Mol. Biol. 355, 619-627 (2006)). Invasin, a 986-amino acid protein anchored to the outer membrane and encoded by the gene inv, promotes uptake into host-cells by binding to β1-integrins and stimulating Rac-1 (Wong et al., Curr. Opin. Microbiol. 8, 4-9 (2005)). As a proof of concept of the therapeutic potential of our synthetic biology chassis, we created Cyborg E. coli BL21 (DE3) cells expressing Invasin and mOrange (Methods Section M1, FIG. 19) and tested if the Cyborg Cells could invade cancer-derived cell lines SH-SY5Y (neuroblastoma) and HeLa (adenocarcinoma) (FIG. 5B-G, Methods Section M14&M15).

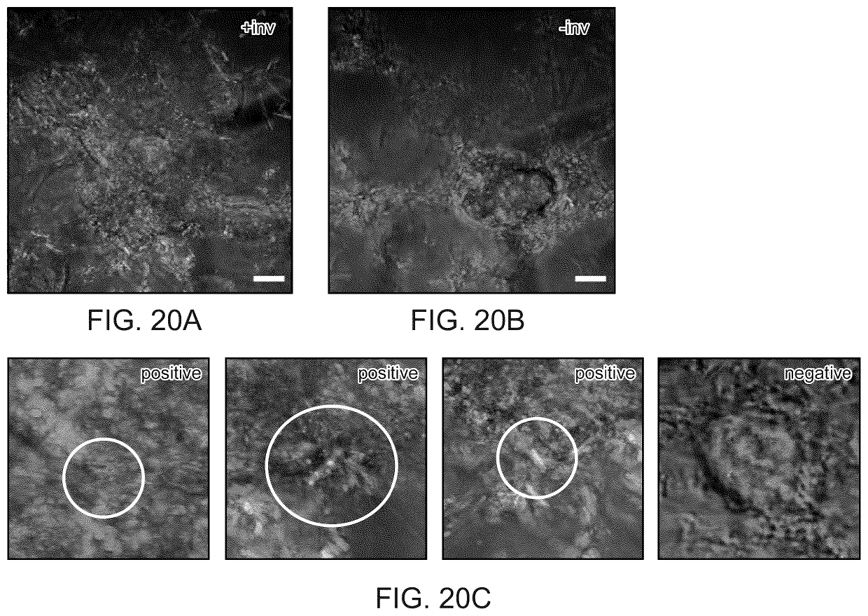

Our in vitro experiments showed that Cyborg Cells expressing Invasin could invade cancer cells. Cyborg Cells expressing Invasin and mOrange were co-incubated with SH-SY5Y cells for four hours (37° C., 5% CO₂). After incubation, Cyborg Cells were washed twice and stained with Hoechst dye. Immediately after, all wells containing SH-SY5Y Cells were imaged using confocal microscopy (Methods Section M14). Confocal microscopy was used to evaluate invasion efficacy because conventional assays that rely on CFU (Leong et al., EMBO J. 9, 1979-1989 (1990)) are not feasible: Cyborg Cells do not replicate. In this experiment, we found distinct differences in the invasion capability of Cyborg Cells expressing Invasin and mOrange vs. Control Cyborg Cells only expressing mOrange (FIG. 5B-D). Cross-sectional images show that Cyborg Cells expressing Invasin were located inside the cellular cytoplasm (FIG. 5E). In addition, we performed a blind counting test of our microscopy images (FIG. 20, Methods Section M14). This assay shows that Cyborg Cells expressing Invasin and mOrange were able to invade ˜34% of SH-SY5Y cells compared to 15% of the SH-SY5Y cells when they were incubated with Cyborg Cells expressing only mOrange (FIG. 5F).

In addition, we tested our Cyborg bacteria expressing Invasin and mOrange with HeLa cells, a patient-derived cell line that has been used to screen for invasive E. coli strains. Similar to our previous experiments, Cyborg Cells expressing Invasin invaded Hela cells after a four-hour incubation at 37° C., 5% CO₂ (FIG. 5G, Methods Section M15). Experiments performed using fixed bacterial cells showed no invasion. Altogether, these results highlight the capacity of Cyborg Cells to act as cancer-invading systems. However, we note that the invasion efficiency of our Cyborg Cells could be improved and enhanced in future work by fine-tuning the quantity of Invasin being expressed and with the addition of cancer-targeting surface proteins.

### Discussion

Our work demonstrates how the combination of synthetic materials and natural cells can create semi-living entities with hybrid characteristics and new capabilities. First, we show the infusion of hydrogel components into bacterial cells. Furthermore, contrary to common expectation, careful control of the intracellular assembly of the synthetic polymeric matrix transforms the host bacteria rather than kills them. In addition, we demonstrate that these new synthetic entities maintain key cellular functions such as protein expression, metabolism, and membrane fluidity while becoming unable to divide. Our hydrogelated bacteria show a different phenotype from their Wild Type counterparts becoming resistant to environmental stressors, including high pH, hydrogen peroxide, and cell-wall targeting antibiotics.

Our research establishes a new paradigm in cellular bioengineering by

demonstrating an intracellular materials-based approach to drive living cells to a state of quasi vita where they retain and gain certain functions within a limited lifespan. Our work opens the door to new questions about the structure of the hydrogel matrix inside bacteria and its interactions with cytoplasmic proteins and the cellular division machinery. These studies may require additional effort to investigate how the hydrogel affects the cell-cycle control of bacteria, particularly on the timing control and coordination of replication events. Despite E. colibeing the best-characterized model organism, the processes regulating its cell division are not yet fully elucidated. Recent experiments support the hypothesis that cell division is regulated by both replication and replication-independent events (Colin et al., Elife. 10, 1-23 (2021)). Therefore, we speculate that the hydrogel matrix may be stopping cell division by either suppressing DNA replication, restricting the increase in cell size, or both. To provide a definitive answer, subsequent work may combine Cryo-EM, high-resolution microscopy, and proteomic analysis at different stages of the lifespan of the Cyborg Cells to reveal localized protein-hydrogel interactions. Altogether, our experiments and these new questions form the basis of a new area that studies the interface between intracellular hydrogels and biomolecules. It is expected Cyborg Cells can be used for in vivo applications, such as antibacterial treatment, biosensors, gut microbiome modulation, and cancer therapy. We envision that our Cyborg Cells would become a new class of synthetic therapy-delivering systems positioned between classical synthetic materials and cell-based systems. The unique set of characteristics of our Cyborg Cells powered by a combination of synthetic biology, materials science, and bioengineering principles may give rise to a new platform to develop novel biotechnological applications.

### Supplementary Materials

### Section A: Methods

### M1: Construction of Plasmids and Strains

Plasmids. In this study, we used the plasmids pLysS (Novagen), and pSC101 (Manen and Caro, Mol. Microbiol. 5, pp. 233-237 (1991)) as the backbones for all our constructs. The backbone of the plasmid pSC101 was used to construct the plasmids plURKL-C.mOrange, and pIURKL. The backbone of the plasmid pLysS was used to construct the plasmids pIURCM and pIURCM-Invasin. All these plasmids have compatible replication origins, distinct copy number, carry a Nsil/Pacl cloning site downstream of a PT7-lacO hybrid promoter, and have a T7RNAP terminator sequence. pIURCM and pIURCM-Invasin contains the chloramphenicol resistance gene/p15A replication origin and expresses T7 lysozyme, pIURKL and pIURKL-C.mOrange contain kanamycin resistance gene/pSC101 replication origin. The plasmids pIURCM, pIURKL and pIURKL-C.mOrange were constructed by Villareal et al. (Villarreal et al., Nat. Chem. Biol. 14, 29-35 (2018)). And are available through Addgene [https://www.addgene.org/Cheemeng_Tan/]. The plasmid pIURCM-Inv was generated by PCR amplifying the inv gene encoding invasin from Yersinia pseudotuberculosis from the plasmid pAC-TetIny (Anderson et al., J. Mol. Biol. 355, 619-627 (2006)) (Gift from Christopher Voigt) and inserting it into the PCR amplified backbone of the plasmid pIURCM using Gibson Assembly (New England Bio-Labs, Inc).

The Marionette Sensor Collection (Meyer et al., Nat. Chem. Biol. 15, 196-204 (2019)) was a gift from Christopher Voigt (Addgene Kit #1000000137). Strains obtained from Addgene were used as the source for the plasmids pAJM.711, pAJM.712, pAJM.713, pAJM.714, pAJM.715, pAJM.717, pAJM.716, pAJM.718, pAJM.719, pAJM.1459, pAJM.721, and pAJM.944. All the plasmids from the Marionette Sensor Collection were used without further modification.

Strains. E. coli Top-10 cells (Thermo Fisher) were used throughout this study for plasmid propagation and maintenance. We used the strains E. coli BL21 (DE3), E. coli Nissle 1917. E. coli MG1655, E. coli (Migula) Castellani and Chalmers (ATCC 25922™) as the main strains for all our experiments. We created a reporter strain by transforming the plasmids pLysS and pIURKL-C.mOrange into E. coli BL21 (DE3). Additionally, we created a cell invasion strain by transforming the plasmids pIURCM-Inv and pIURKL-C.mOrange into E. coli BL21 (DE3). The resulting strains are capable of mOrange expression (reporter strain) and the expression of invasin and mOrange (cell invasion strain) to allow mammalian cell invasion and fluorescent reporting in response to IPTG induction. Both strains have resistance to chloramphenicol (34 μg mL⁻¹) and kanamycin (30 μg mL⁻¹).

For Cyborg Marionette experiments we used the strain Marionette-Pro (sAJM. 1505; Marionette cluster inserted in the direction of leading strand replication, between 3,720,027 and 3,721,644 in E. coli BL21) to transform all twelve plasmids (pAJM.711, pAJM.712, pAJM.713, pAJM.714, pAJM.715, pAJM.717, pAJM.716, pAJM.718. pAJM.719, pAJM.1459, pAJM.721, and pAJM.944) to create Marionette strains responsive to 2,4-diacetylphophloroglucinol (DAPG), cuminic acid (Cuma), 3-oxohexanoyl-homoserine lactone (OC6), vanillic acid (Van), isopropyl β-d-1-thiogalactopyranoside (IPTG), anhydrotetracycline (aTc), 1-arabinose (Ara), choline chloride (Cho), naringenin (Nar), 3,4-dihydroxy benzoic acid (DHBA), sodium salicylate (Sal), and 3-hydroxytetradecanoyl-homoserine lactone (OHC14), respectively. All resulting Marionette strains have resistance to low chloramphenicol (5 μg mL⁻¹) and kanamycin (30 μg mL⁻¹).

M2: Intracellular Hydrogelation of E. coli Cells

All the E. coli strains used in this study were hydrogelated using the same core protocol with modifications to account for specific requirements of individual synthetic modules or proteins being expressed. Each strain was grown overnight in 3 mL of 2YTP media at 37° C. with shaking at 250 rpm and supplemented, if necessary, with chloramphenicol 34 μg mL⁻¹ & kanamycin 30 μg mL⁻¹ (Only for E. coli BL21 (DE3) transformed with the plasmids pIURKL-mOrange and pLysS, & pIURKL-mOrange and pIURCM-Invasin). For Marionette Strains, we used a concentration of chloramphenicol of 5 μg mL⁻¹. The overnight cultures were diluted 5-fold using 2YTP media with the appropriate antibiotics and incubated for ˜2.5 h until the culture reaches an OD of 0.8-1.0. After reaching the appropriate OD, cells are harvested (4000 g, 10 min, 20° C.), resuspended in 2YTP media without antibiotics at a cell density of 0.2 g mL⁻¹, aliquoted into 1.5 mL microcentrifuge tubes (ThermoFisher Scientific), and incubated with hydrogelation buffer (1 WT % 2-hydroxy-4′-(2-hydroxyethoxy)-2-methylpropiophenone (Irgacure D-2959; Sigma-Aldrich), and 5% poly (ethylene glycol) diacrylate (PEG-DA; Mn=700 Da; Sigma-Aldrich) for 30 min at 37° C. with constant rotation at 0.125 Hz on a rotary axis such that the tubes were inverted with each rotation. Fluorescein labeling of the hydrogel polymeric matrix was carried out by supplementing the hydrogelation buffer with 0.1 WT % of fluorescein O,O′-diacrylate (Sigma-Aldrich). After incubation with the hydrogel buffer, bacterial cells were flash frozen by submerging the vials in supercooled methanol at −80° C. for 2 min. Cells were then incubated at −80° C. for 10 min, and then thawed at 30° C. in a dry bath. Vials with bacterial cells were immediately spined down after thawing (6,800 g, 10 min, 20° C.), and washed twice using fresh 2YTP media without antibiotics. Bacterial cells infused with hydrogel were then crosslinked with UV light using an energy delivery of 1600 mJ/cm2 (Light source: UVP Crosslinker CL-3000L—Longwave (365 nm), 115V, Analytik Jena GmbH). Following UV irradiation, cells were spun down (6,800 g. 10 min. 20° C.), and washed twice using 1× PBS buffer. After the final wash and centrifugation, the cells were resuspended and incubated (37° C., constant rotation at 0.125 Hz on a rotary axis) in 2YTP media with the appropriate antibiotics for each strain, plus carbenicillin (400 μg mL⁻¹) to kill actively dividing, non-hydrogelated bacteria. After incubation, the cells were harvested (6,800 g. 10 min, 20° C.) and washed with IX PBS before being resuspended in 2YTP with the appropriate antibiotics for each strain and carbenicillin (100 μg mL⁻¹) for further experiments.

### M3: Detergent Treatment for Hydrogelation Assessment

To assess the successful intracellular hydrogelation of cyborg and wild type cells, we treated different cell populations with 1% Sodium Dodecyl Sulfate (SDS) in PBS buffer to strip cells from their membrane. Briefly, all cells under detergent treatment were washed with PBS buffer twice (6,800 g, 5 min, 20° C.) and resuspended in 1% SDS in PBS buffer. Cells were incubated at room temperature with gentle horizontal shaking (60) rpm) for 10 min. Cells were then spined down (6,800 g, 5 min, 20° C.) and resuspended in PBS buffer without antibiotics before being imaged using fluorescence microscopy.

### M4: Metabolic Activity Assessment

To assess and compare the metabolic activity of different populations of cyborg and wild type cells, we used the cell permeable resazurin-based PrestoBlue™ Cell Viability Reagent (ThermoFisher Scientific).

After hydrogelation, cells were spined down (6,800 g, 5 min, 20° C.) and resuspended in 2YT media with the appropriate antibiotics (chloramphenicol 34 μg mL−1, & kanamycin 30 μg mL−1, & carbenicillin 100 μg mL−1 for hydrogelated bacteria). We plated 90 uL of each sample into a 96 well plate (Corning) and diluted the cells with media with the appropriate antibiotics (Final OD600 of 0.1), then we added the PrestoBlue reagent according to the manufacturer specifications to a final concentration of 1×. The fluorescence intensity of the PrestoBlue reagent (Fluorescence top reading, Excitation 560 nm, Emission 595 nm, gain 100) was monitored every 10 minutes for 3 hours (30° C., double orbital shaking with a frequency of 144 rpms and an amplitude of 2.5 mm, 60 s ON, 540 s OFF) using an m1000Pro Infinite plate reader (Tecan). Each one of these experiments was compared against a “Wild Type” strain consisting of non-hydrogelated bacteria with the same phenotype as the Cy borg Cells.

### M5: Fluorescence Microscopy

Microscope images were recorded using a Nikon Eclipse Ti inverted fluorescence microscope with perfect focus 3 (Nikon Instruments Inc) equipped with a 100×/1.4 oil objective. Exposure times were Fluorescein, 300 ms; mOrange 300 ms; Bright Field, 56 ms. Microscope filter settings were Fluorescein, excitation, 450-490 nm; emission, 500-550 nm; gain ≥495 nm. mOrange, excitation, 532-557 nm; emission, 570-640 nm; gain ≥565. Samples of cyborg cells, and different bacterial controls were imaged using lab-made 1.5% Low Melting Temperature Agar (LMTA), 1× PBS gel pads. These lab-made gel pads (50×25 mm, Thickness: 1 mm) were mounted over glass slides (Plain Micro Slides, 75×50 mm, Thickness 1 mm, Corning Inc) and divided in 8 individual squares to allow for imaging of 8 separate samples at once.

Overnight fluorescence microscopy experiments were carried out using similar lab-made 1.5% LMTA, 2YT or 1× PBS gel pads with one isolated square in the center of the gel pad and the rest of the hydrogel arranged to protect the center square from shrinking due to evaporation. Overnight tracking was carried out by taking a 3×3 image every 20 minutes (30° C.).

Quantification of fluorescence expression was carried out by measuring the pixel intensity of cyborg bacterial cells and controls in the mOrange fluorescence channel using the open-source platform for biological imaging analysis Fiji (http://fiji.sc/cgi-bin/gitweb.cgi/).

### M6: Interior Hydrogelation Gel of Porous Structure Using Cryo Scanning Electron Microscope (Cryo-SEM)

For obtaining the porous structure image of interior gel, gel was freshly prepared, dissected, and mounted on a stub. The gel was frozen with liquid nitrogen, and then transferred to a preparation chamber at −160° C. As the temperature had reached around −130° C., the sample was etched at −85° C. for 15 min. After coating with platinum at −130° C., the sample was transferred to the SEM chamber and under vacuum at −180° C. with an acceleration voltage of 20 kV using a cryo scanning electron microscope (FEI Quanta 200/Quorum U.S. Plant Pat. No. 2,000TR FEI).

### M7: FRAP Analysis

Cell membrane was stained by adding 10 uL of DiD dye solution (1,1′-Dioctadecyl-3,3,3′,3′-tetramethylindodicarbocyanine; ThermoFisher Scientific) containing 5 ugmL−1 of DiD dye and 0.5% of DMSO to 200 μL of cell suspension. E. coli BL21 (DE3) pLysS pIURKL-mOrange cells were grown overnight and were either hydrogelated or treated with 2.5% glutaraldehyde for 10 min prior to FRAP analysis. FRAP analysis was carried out on a Zeiss LSM780 confocal microscope (Carl Zeiss, Oberkochen, Germany) equipped with Plan-Apochromat 100×/1.4 oil objective. For FRAP analysis, adherent cells and adherent GCs were used rather than suspension cells to minimize artifacts due to random movements. An objective heater was used to maintain samples at 37° C. Images were collected with a pinhole of 1.52 AU (1.1 μm section) for optimal signal intensity. The sample was first scanned three times with 5% of laser power to measure the fluorescence intensity before photobleaching, followed by 500 iterative laser pulses at full power to photobleach a 27 nm×6 nm rectangular area at the plasma membrane. Fluorescence recovery was monitored every 2 s for at least 2 min at 60 frames per second until a plateau is reached. Fluorescence intensity vs. time was plotted for analyzing the fluorescence recovery.

### M8: CFU Assay

After intracellular hydrogelation of E. coli cells, we assessed the proliferation capabilities of the resulting cell population by short and long term CFU assays. Briefly, the resulting cells were diluted 10⁻²-10⁻⁸ times and 20 uL were plated in triplicate into individual wells of 24 well plates (Corning) with 2YT agar with the appropriate selection markers. Plates were incubated overnight (37° C.) and the resulting colonies in the appropriate dilutions were counted and reported as CFUs accounting for the dilution factor and the volume used to plate. For accuracy and consistency, we only counted colonies in a dilution if there were between 5-50 colonies. A result of less than 5 colonies was considered as below the detection level.

### M9: Motility Assay

After intracellular hydrogelation of different E. coli populations, we visualized the motility of the resulting Cyborg Cells and their Wild Type controls using Bright Field microscopy. Microscope images were recorded using a Nikon Eclipse Ti inverted fluorescence microscope with perfect focus 3 (Nikon Instruments Inc) equipped with a 100×/1.4 oil objective. Following standard hydrogelation, we resuspended our samples in 1× PBS and standardized the OD600 to 0.1. Next, we took 50 uL of each sample and plated them individually on separate wells of an ibidi 15 μ-Slide Angiogenesis (IBIDI GMBH). After allowing for cells to settle down for 20 minutes, we captured Fast Timelapses of the samples in focus with the maximum frame number (100) Frames). We tracked the movement in the field of view of individual moving cells across each one of the 100 frames and representative frames with tracked cells were selected to illustrate the motility of different samples. Image analysis was carried out using the open-source platform for biological imaging analysis Fiji (http://fiji.sc/cgi-bin/gitweb.cgi/). We assembled videos using all frames of each experiment and the resulting files are showing as supplementary materials for this manuscript.

### M10: Response of Cyborg Marionette Cells to Different Inducers

We used the Marionette Sensor Collection from Addgene (Addgene Kit #1000000137) (Meyer et al., Nat. Chem. Biol. 15, 196-204 (2019)) to create 12 different strains responsive to the 12 small molecules DAPG, Cuma, OC6, Van, IPTG, aTc, Ara, Cho, Nar, DHBA, Sal, and OHCl4. The strains were built by transforming the Marionette protein expression strain Marionette-Pro with each one of the plasmids containing reporters to each one of those 12 small molecules (Methods Section M1). These strains have been shown to express the fluorescent reporter YFP in response to the appropriate inducer.

To create Cyborg Marionette Bacteria, each strain was hydrogelated based on the standard hydrogelation protocol described in “Methods Section M2” with a small variation, the hydrogel polymeric matrix is not labeled with fluorescein due to the fluorescence overlap with YFP (Fluorescein; Excitation max 490 nm, Emission max 525 nm. YFP; Excitation max 513 nm, Emission max 527 nm). Immediately after hydrogelation, cells were spun down (6,800 g. 5 min. 20° C.) and resuspended in 2YT media with the appropriate antibiotics (chloramphenicol 5 μg mL⁻¹, & kanamycin 30 μg mL⁻¹, & carbenicillin 100 μg mL⁻¹ for hydrogelated bacteria) and if required with the appropriate inducers (Table 1) to an OD600 of 0.1. We plated 100 uL of each sample into a 96 well plate (Corning) and the YFP fluorescence intensity (Fluorescence top reading, Excitation 510 nm, Emission 525 nm, gain 100) was monitored every 5 minutes for 12 hours (30° C., double orbital shaking with a frequency of 120 rpms and an amplitude of 3 mm, 60 s ON, 240 s OFF) using an m1000Pro Infinite plate reader (Tecan). Each one of these experiments was compared against a “Wild Type” Marionette strain consisting of non-hydrogelated bacteria.

M11: mOrange Expression Tracking

To track the expression of mOrange by Cyborg and Wild Type Cells, we used the strain E. coli BL21 (DE3) pLysS pIURKL-mOrange (Methods Section M1). We plated 100 uL of hydrogelated and Wild Type cells into a 96 well plate (Corning) and the mOrange fluorescence intensity (Fluorescence top reading, Excitation 548 nm, Emission 562 nm, gain 100) and the absorbance at 600 nm was monitored every 5 minutes for 12 hours (30° C., double orbital shaking with a frequency of 216 rpms and an amplitude of 3 mm, 120 s ON, 180 s OFF) using an m1000Pro Infinite plate reader (Tecan).

### M12: Mass Spectrometry

The following protocol was used for initial sample processing, and it was intended to produce a cell free lysate from each one of the samples to facilitate the downstream workflow for mass spectrometry: After hydrogelation (or treatment according to each control, see Note below), Cyborg and bacterial cells were harvested and washed twice with 1 mL of Buffer A (4000 g, 20 min, 4°° C.). Buffer A contains 10 mM Tris-acetate pH 7.6, 14 mM magnesium acetate, and 60 mM potassium gluconate. After the final wash and centrifugation, the pelleted cells were weighed and suspended in 1 mL of Buffer A supplemented with 2 mM DTT (Thermo Fisher Scientific) per 1 g of wet cell mass. To lyse cells by sonication, freshly suspended cells were transferred into 1.5 mL microtubes and placed in an ice-water bath to minimize heat damage during sonication. The cells were lysed using a Q125 Sonicator with a 2 mm diameter probe at a frequency of 20 KHz and 50% amplitude. Sonication was continued for about 27 cycles 10 s ON/10 s OFF. For each 0.5 mL sample, the input energy was ˜1000 J. Cell lysates were centrifuged at 12,000 g for 20 min at 4° C. The supernatant was collected and stored at −80° C. until further use for peptide sample preparation.

The following protocol was used for peptide sample preparation: the proteins in the whole-cell extract preparations were quantified using BCA assay (Thermo Scientific). A volume equal to 250 μg of protein was used for S-Trap (PROTIFI) digestion. Digestion followed the S-trap protocol; briefly, the proteins were reduced and alkylated, the buffer concentrations were adjusted to a final concentration of 5% SDS 50 mM TEAB, 12% phosphoric acid was added at a 1:10 ratio with a final concentration of 1.2% and S-trap buffer (100 mM TEAB in 90% MeOH) is added at a 1:7 ratio (V/V ratio). The protein lysate S-trap buffer mixture was then spun through the S-trap column and washed 3 times with S-Trap buffer. Finally, 50 mM TEAB with 6 μg of trypsin (1:25 ratio) is added and the sample is incubated overnight with one addition of 50 mM TEAB with trypsin after 2 h. The following day the digested peptides were released from the S-trap solid support by spinning at 3000 g for 1 min with a series of solutions starting with 50 mM TEAB which is placed on top of the digestion solution, then 5% formic acid followed by 50% acetonitrile with 0.1% formic acid. The solution is then vacuum centrifuged to almost dryness and resuspended in 2% acetonitrile 0.1% Triflouroacetic acid and subjected to fluorescent peptide quantification (Thermo Scientific).

The following protocol was used for peptide labeling with TMTs and fractionation: A set of TMTpro-16plex labels were used to label the samples. In total 25 μg of each sample was diluted with 50 mM TEAB to 25 μL per replicate. Each sample was labeled with the TMTpro-16Plex Mass Tag Labeling Kit (Thermo Scientific). Briefly, 20 μL of each TMTpro label (126-134N) was added to each digested peptide sample and incubated for an hour. The reaction was quenched with 1 μl of 5% hydroxylamine and incubated for 15 min. All labeled samples were then mixed and lyophilized to almost dryness. The TMTpro labeled sample was reconstituted, desalted, and separated into eight fractions by high pH fractionation (Thermo

Scientific). One-third of each fraction (˜800 ng) was loaded on to the LC-MS/MS for analysis.

The following protocol was used for liquid chromatography and mass spectrometry of the samples: liquid chromatography separation was conducted on a Dionex nano Ultimate 3000 (Thermo Scientific) with a Thermo Easy-Spray source. The digested peptides were reconstituted in 2% acetonitrile/0).1% trifluoroacetic acid and 1 μg in 5 μL of each sample was loaded onto a PepMap 100 Å 3U 75 μm×20 mm reverse-phase trap where they were desalted online before being separated on a 100 A 2U 50 μm×150 mm PepMap Easy Spray reverse phase column. Peptides were eluted using a 120-min gradient of 0.1% formic acid (A) and 80% acetonitrile (B) with a flow rate of 200 nL/min. The separation gradient was run with 2-5% B over 1 min, 5-50% B over 89 min, 50-99% B over 2 min, a 4-min hold at 99% B, and finally 99% B to 2% B held at 2% B for 18 min.

The following protocol was used for mass spectra acquisition: mass spectra were collected on a Fusion Lumos mass spectrometer (ThermoFisher Scientific) in a data-dependent MS3 synchronous precursor selection method. MSI spectra were acquired in the Orbitrap, 120 K resolution, 50 ms max injection time, 5×105 max injection time. MS2 spectra were acquired in the linear ion trap with a 0.7 Da isolation window, CID fragmentation energy of 35%, turbo scan speed, 50 ms max injection time, 1×104 AGC, and maximum parallelizable time turned on.

MS2 ions were isolated in the ion trap and fragmented with an HCD energy of 65%. MS3 spectra were acquired in the orbitrap with a resolution of 50 K and a scan range of 100-500 Da, 105 ms max injection time, and 1×105 AGC.

The following process was followed for peptide and protein identification: identification of peptides and proteins was conducted using the PAW pipeline (Wilmarth et al., J. Ocul. Biol. Dis. Infor. 2, 223-234 (2009)). In brief, the ProteoWizard toolkit is used to convert the MS scans into intensity values and extract the TMT reporter ion peak heights. The Comet database search engine is then used to identify peptides. The E. coli BL21 (DE3) proteome UP000002032 and a list of known contaminants and expressed protein sequences were used for protein identification. Results are filtered based on a desired false discovery rate using the target decoy method. Identified proteins with sequence coverage of <5% were excluded from the downstream analysis.

The following process was used to normalize the TMT results to account for differences in sample loading: The protein intensities from each of the tagged samples were summed and the average of all the sums was calculated. A normalization factor was calculated by dividing the average sum by the sum of the sample. The normalization factor for each sample was then multiplied by all the protein intensities present.

The following process was used for the assignment of gene ontological function: identified proteins were assigned gene ontological functions based on the gene ontology identifiers provided in the E. coli BL21 (DE3) proteome UP000002032. The gene ontology identifiers were grouped based on the general functional categories of interest. Proteins that lacked identifiers or only possessed broad identifiers were classified as Unknown. Several proteins with known functions that lacked identifiers were manually assigned an appropriate functional group.

Note: The control cells for this experiment were obtained by following the standard hydrogelation procedure described in Methods Section M2 but omitting steps or incubation with certain reagents to create non-hydrogelated cells subjected to only specific parts of the hydrogelation procedure. The “UV-Treated control” cells were obtained by omitting the incubation with hydrogel buffer and the incubation with a high concentration of Carbenicillin. The “HG-treated control” cells were obtained by omitting the crosslinking with UV light and the incubation with high concentration of Carbenicillin. The “Wild Type control” cells were obtained by omitting the incubation with hydrogel, the exposure to UV light and the incubation with high concentration of carbenicillin.

### M13: SDS-PAGE Analysis

Analysis of proteins by SDS-Polyacrylamide Gel Electrophoresis (PAGE) was carried out by separating proteins from whole-cell lysates and CFPS reactions using 4-20% Mini-PROTEAN TGX precast gels (Bio-Rad). We used Precision Plus Protein Dual Color Standards (10)-250 kDa) as a reference standard for molecular weight verification. Protein gels were endpoint stained using PageBlue Protein Staining Solution (Thermo Fisher Scientific) according to the manufacturer instructions. Gels were imaged using a PXi Imaging system (Syngene) and band analysis and protein quantification were carried out using the open-source platform for biological imaging analysis Fiji (http://fiji.sc/cgi-bin/gitweb.cgi/) and the proprietary software GeneTools (Syngene).

M14: Invasion of SH-SY5Y Cells by Cyborg and Wildtype E. coli Cells

We used SH-SY5Y Cells (CRL-2266™; SH-SY5Y cells are a thrice cloned subline of the neuroblastoma SK-N-SH line derived from a metastatic bone tumor) as a model for mammalian cancer cells for invasion experiments using cyborg E. coli cells.

SH-SY5Y cells were expanded for said experiments by first taking aliquot of cells from liquid nitrogen tanks and thawing in 37° C. bead bath until thawed. Immediately, 1 mL of cells were resuspended in 4 mL of D5GF media and centrifuged at 1400 RPM for 5minutes. D5GF media comprised of DMEM High Glucose, Fetal Bovine Serum, Epidermal growth factor, fibroblast growth factor, and penicillin-streptomycin. After centrifugation, cell waste was aspirated, and the cell pellet was resuspended in 2 mL of media. A 20 uL aliquot was taken and mixed with 20 uL of trypan blue and counted on a hemocytometer. Live cell averages of the 4 corners of the hemocytometer were taken and used to calculate total live cell count. Cells were then plated on a T25 flask at a density of 5, 000, 000 cells. Cells were grown to confluency with media changes every other day then passaged. The cells were removed from the plate surface by first washing with PBS, then incubating with trypsin for 5 minutes. Trypsin was inactivated using penicillin-streptomycin free D5GF. Cells were centrifuged and counted in the same manner as before. The SH-SY5Y cells were plated on an ibidi μ-Slide 8 Well high Glass Bottom (IBIDI GMBH) at a density of 50,000 cells per well with penicillin-streptomycin free D5GF. Cells were incubated at 37° C. 5% CO₂ for at least 1 day. After 2 days, the cells were washed with pen-free D5GF and incubated with cyber bacteria at a normalized optical density of 0.05 and 0.1 at 37° C. 5% CO₂.

After the 4 h incubation with the cyborg and the Wild Type bacterial cells, SH-SY5Y Cells were washed twice using 1× PBS buffer and stained with Hoechst dye (20 mM, 15 min) for staining of DNA and nuclei of the mammalian cells. Immediately after, all wells containing SH-SY5Y Cells were imaged using confocal microscopy (ZEISS LSM800, 63× objective) and the image analysis was done using ZEISS ZEN Software. For the experiments conducted with the Zeiss Confocal Microscope, mOrange was imaged using an excitation of 546 nm, an emission of 562, a detection wavelength range of 535-700, and a pinhole of 0.83 AU with a laser wavelength of 561 nm at 2.01%. Hoechst was imaged using excitation of 353 nm, emission of 465 nm, a detection wavelength of 400-545/550 and a pinhole of 0,76 AU with a laser wavelength of 405 nm at 0.2%. All experiments were carried out in duplicate with three technical replicates each.

Blind counting of Cyborg Cells invading mammalian cells. To assess the invasion of mammalian cells by cyborg cells, we selected 24 images. (6 representative images per condition), and these were re-named and randomized. The brightfield of the images was artificially colored in green to help with contrast. Two volunteers were selected and were told what each of the stains represented, such as blue for Hoechst, green for brightfield, orange for bacterial cells expressing mOrange. Three example images of invasion and one of non-invasion were shown and explained to each of the volunteers. Individually, the volunteers went through all images and were asked to count number of mammalian cells, number of mammalian cells with at least one bacterium invaded, and total number of bacteria invaded. These numbers were recorded by the volunteers and were hidden from the other volunteer.

M15: Invasion of HeLa Cells by Cyborg E. coli Cells

Hela cells (CCL-2™; Hela cells are cervical carcinoma cell line derived from a patient) were grown in a media comprised of DMEM (Life Technologies, cat. 11995065), Fetal Bovine Serum (FBS), and Penicillin-Streptomycin (PS, Life Technologies, cat. 15140-122) and plated on a confocal dish at a density of 2×10⁵ cells/well. The cells were grown overnight at 37° C., with 5% CO₂. The culture medium was removed. Cells were stained with 20 μM of Celltracker Blue CMAC dye (Invitrogen, CA) in DMEM only (without FBS and PS) for 0.5 hour. After 0.5 h incubation, the DMEM was removed and fresh DMEM was added (with FBS and PS). Bacteria, controls, and Cyborg bacteria were spun down at 4000 l for 10 minutes, and the pellet was resuspended in the Penicillin Streptomycin absent media and mixed with Kanamycin (1:1000 dilution) (VWR cat. 25389-94-0), Chloramphenicol (1:3000 dilution) (AMRESCO cat. 56-75-7), Carbenicillin (1:1000 dilution) (Cyborg bacteria only) (Millipore Sigma cat. C3416-5G), and 1 mM IPTG. The dosing of the bacteria and Cyborg bacteria was performed using 20 uL of the bacteria, hydrogelated bacteria, or fixed bacteria-media mixture and added to plates containing the mammalian cells placed in the 37° C., 5% CO₂ incubator. The bacteria were incubated for 4 hours together with the mammalian cells. After this incubation, the wells were washed twice with DMEM and then washed once with PBS (Fisher Scientific, cat. BP243820). The cells were kept in 4% paraformaldehyde/PBS for imaging. Imaging was performed using confocal microscopy (ZEISS LSM880, 63× objective) and image analysis using the software ZEISS ZEN.

### M16: Statistical Analysis of Results

Statistical tests were performed using standard two-tailed t-test, assuming unequal variances. Significant results were defined as those with p-values less than 0.05 and were indicated in each figure by adding an asterisk. The number of replicates contributing to the calculation is listed in the figure legends.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A metabolically-active cell comprising a cross-linked hydrogel within the cell in sufficient amount to prevent cell replication.

2. The metabolically-active cell of claim 1, wherein the hydrogel comprises monosaccharide or polysaccharide monomer subunits and wherein the hydrogel is a homopolymer or co-polymer.

3. The metabolically-active cell of claim 2, wherein the hydrogel comprises substituted or unsubstituted poly (ethylene glycol) monomer subunits.

4. The metabolically-active cell of claim 2, wherein the hydrogel comprises poly (dimethyl siloxane) (PDMS), poly (ethylene oxide) (PEO), poly (vinyl alcohol) (PVA), poly (propylene fumarate) (PPF), alginate, guanosine mono phosphate (GMP), cyclodextrin (CD), fibrin, collagen, polypeptides, decellularized extracellular matrix, or nucleic acids.

5. The metabolically-active cell of any one of claim 1-4, wherein the hydrogel is substituted.

6. The metabolically-active cell of claim 5, wherein the hydrogel is conjugated to a metal, bioactive or therapeutic molecule, drug, nanoparticle, nucleic acid, or polypeptide.

7. The metabolically-active cell of claim 6, wherein the bioactive molecule is an anti-cancer molecule.

8. The metabolically-active cell of claim 1, wherein the hydrogel has a density of 1-10% (w/w) in the cell.

9. The metabolically-active cell of claim 1, wherein the cells are prokaryotic cells.

10. The metabolically-active cell of claim 9, wherein the prokaryotic cells are gram negative bacteria.

11. The metabolically-active cell of claim 10, wherein the gram-negative bacteria are selected from the genera consisting of Escherichia, Proteus, Enterobacter, Klebsiella, Citrobacter, Yersinia, Shigella, and Salmonella.

12. The metabolically-active cell of claim 1, wherein the cells are eukaryotic cells.

13. The metabolically-active cell of claim 12, wherein the cells are eukaryotic cells are yeast or plant or mammalian cells.

14. The metabolically-active cell of claim 12, wherein the eukaryotic cells are Saccharomyces cerevisiae cells.

15. The metabolically-active cell of claim 12, wherein the mammalian cells are HeLa, HEK293, or SH-SY5Y cells.

16. The metabolically-active cell of claim 1, further comprising at least one heterologous nucleic acid.

17. The metabolically-active cell of claim 16, wherein the heterologous nucleic acid encodes a protein.

18. The metabolically-active cell of claim 17, wherein the protein is an enzyme.

19. The metabolically-active cell of claim 1, wherein the cell has been modified to have a reduced amount of one or more nuclease, protease and protein involved in stress response compared to a native cell.

20. The metabolically-active cell of claim 1, wherein the cell is contacted with a heterologous cryoprotectant.

21. The metabolically-active cell of claim 1, wherein the cell is modified with a heterologous molecule that directs flux of ATP and/or NADH.

22. A method, comprising administering the cells of any one of claims 1-21 to an animal.

23. The method of claim 22, wherein the animal is human.

24. A method of assaying a cellular activity of the cells of any one of claims 1-21, the method comprising, measuring at least one activity of the cells

25. The method of claim 24, wherein the measuring comprises contacting the cells with an agent and measuring the effect of the agent on the activity of the cells.

26. The method of claim 24, wherein the activity is selected from the group consisting of cellular motility, intracellular redox (reduction/oxidation) state, membrane fluidity, and protein expression capabilities.

27. A method of generating the cell of any one of claims 1-21, the method comprising,
providing dividing cells;
introducing monomer units of a hydrogel into the cells;
causing the polymerization inducer to initiate formation in the cells of a hydrogel formed from the monomer units thereby forming a mixture of cells comprising the hydrogel.

28. The method of claim 27, further comprising introducing a polymerization inducer into the cells before, after or simultaneously with the introducing of the monomer units.

29. The method of claim 28, wherein the polymerization inducer is activated by light of a specific wavelength and the causing comprising exposing the cells to light of the specific wavelength.

30. The method of claim 29, wherein the monomer subunits comprise one or more acrylate moieties and the polymerization inducer is selected from the group consisting of 2-hydroxyl-4′-(2-hydroxyethoxy)-2-methylpropiophenone, Irgacure 2959, Eosin-Y, and lithium phenyl-2,4,6-tri-methylbenzoylphosphinate.

31. The method of any one of claims 28-30, wherein the monomer subunits comprise substituted or unsubstituted poly (ethylene glycol) monomer subunits.

32. The method of claim 31, wherein the substituted or unsubstituted poly (ethylene glycol) monomer subunits comprise poly (ethylene glycol) diacrylate, poly (ethylene glycol) thiol poly (ethylene glycol) vinyl sulfone, alginate, guanosine mono phosphate (GMP), cyclodextrin (CD), fibrin, collagen, polypeptides, decellularized extracellular matrix, or nucleic acids.

33. The method of claim 27, wherein the monomer subunits comprise poly (dimethyl siloxane) (PDMS), poly (ethylene oxide) (PEO), poly (vinyl alcohol) (PVA), or poly (propylene fumarate) (PPF).

34. The method of claim 27, wherein the introducing comprises exposing the cells to a freeze/thaw cycle in the presence of the monomer units and the polymerization inducer.

35. The method of any one of claims 27-34, further comprising contacting the mixture of cells with a replication-specific toxin and/or antibiotics, thereby killing cells in the mixture capable of replicating.

36. The method of any one of claims 27-35, further comprising contacting the cell with a heterologous cryoprotectant during the providing, introducing and/or causing.
